(19) United States
(12) Patent Application Publication
(10) Pub. No.: US 2008/0009639 A1
(43) Pub. Date: Jan. 10, 2008

(54) Preparation of oseltamivir phosphate (Tamiflu) and intermediates starting from D-glucose or D-xylose

(75) Inventors: Bruno Konrad Radatus, Brantford (CA); K.S. Keshava Murthy, Ancaster (CA); Gamini Weeratunga, Brantford (CA); Stephen E. Horne, Burlington (CA); Kiran Kumar Kothakonda, Brantford (CA); Eckardt C.G. Wolf, Brantford (CA); Zhongyi Wang, Guelph (CA)

Correspondence Address: IVOR M. HUGHES, BARRISTER & SOLICITOR,;PATENT & TRADEMARK AGENTS, 175 COMMERCE VALLEY DRIVE WEST, SUITE 200, THORNHILL, ON, L3T 7P6, US

(73) Assignee: Apotex Pharmachem Inc.

(21) Appl. No.: 11/709,658

(22) Filed: Feb. 23, 2007

Related U.S. Application Data
(60) Provisional application No. 60/819,365, filed on Jul. 10, 2006.
(60) Provisional application No. 60/898,464, filed on Jan. 31, 2007.

(51) Int. Cl. C07C 229/00; C07C 311/01; C07D 203/26; C07D 307/02; C07F 7/04; C07F 9/28

(52) U.S. Cl. 548/961; 549/220; 549/221; 549/429; 556/449; 558/56; 560/125

(57) ABSTRACT

Novel processes for the preparation of the anti-viral agent, Oseltamivir Phosphate and novel intermediates prepared in such processes. The novel processes use as starting materials D-glucose or D-xylose in the preparation of Oseltamivir Phosphate.

### CROSS REFERENCE TO RELATED APPLICATIONS

This Application claims the benefit of U.S. Provisional Application No. 60/819,365 filed on Jul. 10, 2006 and U.S. Provisional Application No. 60/898,464 filed on Jan. 31, 2007.

### FIELD OF THE INVENTION

The present invention relates to a new and cost-effective process for the manufacture of novel intermediates, useful for the preparation of Oseltamivir Phosphate (Tamiflu®) from abundant and inexpensive carbohydrate precursors and processes to prepare Oseltamivir Phosphate.

### BACKGROUND OF THE INVENTION

Avian flu “bird flu”, which is now considered to be endemic in birds in large parts of China, is caused by the H5N1 influenza virus. By the end of May 2006, as reported by the World Health Organization (WHO), the virus had spread westward to Europe among birds and has infected 218 humans in one African and nine Asian countries by intimate contact with infected birds and human to human contact which has resulted in 124 human deaths. It is feared that the deadly virus could mutate so that it could spread easily by human to human contact, thereby potentially causing a global pandemic, similar or worse than the pandemic of 1918. Oseltamivir phosphate (Tamiflu®, FIG. 1) has been shown to be effective against human and avian H5N1 influenza viruses and it was suggested that stockpiling would be a prudent course of action in preparation for the possible next pandemic.

In the discovery route for the preparation of Oseltamivir phosphate, (−)-quinic acid was the starting raw material (S. Abrecht et al., Chimica (2004) 58, 621). Subsequently, routes were devised starting from (−)-shikimic acid employing some of the chemistry devised for the quinic acid route, followed by further improvements which focused on the introduction of the 3-pentyloxy group, and culminated in what could be considered as a “first generation” industrial process for the production of Tamiflu® (M. Federspiel et al., Org. Proc. Res. Dev., (1999) 3, 226). Further improvements focused on avoiding the use of azides for the introduction of the nitrogen moieties and still further improvements culminated in a “second generation” industrial Tamiflu® process (P. J. Harrington et al., Org. Proc. Res. Dev., (2004) 8, 86). The preferred starting material for all of the improved processes is (−)-shikimic acid which means that the overall viability of the above industrial processes depend on the cost and availability of this natural product.

Commercial quantities of (−)-shikimic acid are obtained by extraction of the fruit of the star anise plant which is grown in four provinces in China and is harvested between March and May. Significantly, 90% of the harvest is consumed by Hoffmann-La Roche (Roche) and therefore, availability is a problem for new manufacturers of Tamiflu®. Another source for the acid is a biocatalytic process which was developed at Michigan State University (East Lansing) in collaboration with Roche with the carbon source being glucose. The disadvantage of this approach is that biocatalytic expertise may have to be acquired and new investments may have to be made in plant equipment. Achiral starting materials have also been employed such as acrylates with furans using Diels-Alder chemistry, or substituted aromatic compounds where the aromatic rings need to be reduced. The disadvantages of these approaches are the need to perform resolutions and desymmetrizations, respectively. Carbohydrates have been used as chiral pools for the preparation of (−)-shikimic acid with the best yields (29-39%) being obtained when the starting carbohydrate was D-mannose. While starting from mannose initially looks attractive, its cost of about 50 dollars per kilogram makes this approach expensive.

Therefore, a new and industrially acceptable synthesis of Oseltamivir from readily available and inexpensive precursors was highly desirable.

### SUMMARY OF THE INVENTION

According to one aspect of the invention, certain inexpensive and readily available carbohydrates can be used for the preparation of intermediates useful for the preparation of Tamiflu®. These include D-glucose and D-xylose. D-glucose and D-xylose are the least expensive of the hexose and pentose families, respectively. D-xylose is marginally more expensive than D-glucose.

Surprisingly the C1-C5 carbons of D-glucose or D-xylose have been elaborated by us to the C2-C6 carbons (conventional numbering) of a key Oseltamivir intermediate 6 (compound 6). Furthermore as depicted in FIG. 2, when the stereochemical configurations at carbons C2-C4 of D-glucose or D-xylose are inverted, they yield the desired configurations for carbons C3-C5 of Oseltamivir. Thus we can establish in our intermediates the requisite stereochemistry of Oseltamivir after the appropriate manipulations of the chirality of the starting carbohydrates.

According to one aspect of the invention, Compound 6 is obtained from the process according to the present invention as illustrated in FIG. 3.

The starting point is the conversion of D-glucose or D-xylose to compound 1 by processes known in the prior art such as R. L. Whistler et al., J. Org. Chem., (1972) 37, 3187, P. Strazewski et al., Tetrahedron (1998) 54, 13529 and O. Midraoka et al., Bioorganic & Medicinal Chemistry (2006), 14, 500. For instance, starting from glucose, the diacetonide is prepared followed by triflation, azide displacement, hydrolysis of the primary acetonide, periodate cleavage to the aldehyde, and sodium borohydride reduction to the alcohol. Subsequently, the primary hydroxyl on compound 1 is converted into a leaving group (Lv) to form Compound 2. The leaving group is then displaced from Compound 2 to form Compound 4. Compound 4 is subsequently hydrolyzed to Compound 5. Compound 5 is cyclized to form Compound 6 by base treatment.

According to a further aspect of the invention, there is provided a process of converting compound 6 to Oseltamivir.

According to yet another aspect of the invention, there is provided a process for the preparation of compound 6, comprising the steps of:
i) converting compound 5 to compound 6 by base treatment to achieve intramolecular cyclization.

According to yet another aspect of the invention, there is provided a process for the preparation of compound 6, comprising the steps of:
i) converting compound 5 to compound 6 by base treatment to achieve intramolecular cyclization;
ii) converting compound 4 to compound 5 by hydrolysis; and
iii) converting compound 2 to compound 4 by displacing the leaving group using an anion of a trialkylphosphonoacetate.

According to yet another aspect of the invention, there is provided a process for the preparation of compound 6, comprising the steps of:
i) converting compound 5 to compound 6 by base treatment to achieve intramolecular cyclization;
ii) converting compound 4 to compound 5 by hydrolysis;
iii) converting compound 2 to compound 4 by displacing the leaving group using an anion of a trialkylphosphonoacetate;
iv) converting compound 1 to compound 2 by converting the primary hydroxyl to a leaving group; and either
v) (a) converting D-glucose to compound 1 by acetonide formation, triflation, azide displacement, acetonide hydrolysis, periodate cleavage, and reduction,
   (b) or converting D-xylose to compound 1 by diacetonide formation, selective acetonide hydrolysis, protection of the primary hydroxyl group, triflation, azide displacement, and removal of the primary hydroxyl protecting group.

According to another aspect of the invention, Compound 10 is obtained from the process according to the present invention as illustrated in FIG. 4.

Compound 1 is converted to compound 7. A solution of triethyl phosphonoacetate, a suitable base, and a crown ether in a suitable organic solvent, was prepared and added slowly to a solution of compound 7 in N,N-dimethylformamide at room temperature. After the reaction was complete, it was quenched with a suitable quenching agent and compound 8 was isolated after work-up. Compound 8 was hydrolyzed to compound 9 using an acid. Compound 9 was then purified. Compound 9 was cyclized by adding a base to yield a solution of compound 10 in an anhydrous solvent. After reaction completion, it yields compound 10.

According to yet another aspect of the invention, there is provided a process for the preparation of compound 10, comprising the steps of:
i) converting compound 9 to compound 10 by base treatment to achieve intramolecular cyclization.

According to yet another aspect of the invention, there is provided a process for the preparation of compound 10, comprising the steps of:
i) converting compound 9 to compound 10 by base treatment to achieve intramolecular cyclization;
ii) converting compound 8 to compound 9 by acid hydrolysis; and
iii) converting compound 7 to compound 8 by displacement of the triflate group using the anion of a dialkylphosphonoacetate.

According to yet another aspect of the invention, there is provided a process for the preparation of compound 10, comprising the steps of:
i) converting compound 9 to compound 10 by base treatment to achieve intramolecular cyclization;
ii) converting compound 8 to compound 9 by acid hydrolysis;
iii) converting compound 7 to compound 8 by displacement of the triflate group using the anion of a dialkylphosphonoacetate;
iv) converting compound 1 to compound 7 by triflation of the primary hydroxyl; and either
v) (a) converting D-glucose to compound 1 by acetonide formation, triflation, azide displacement, acetonide hydrolysis, periodate cleavage, and reduction,
   (b) or converting D-xylose to compound 1 by diacetonide formation, selective acetonide hydrolysis, protection of the primary hydroxyl group, triflation, azide displacement, and removal of the primary hydroxyl protecting group.

According to a further aspect of this invention, there is provided a general process for elaborating compound 10 (or the corresponding compound of general formula 6) to Oseltamivir as illustrated in FIG. 5, FIG. 6 and FIG. 7.

Compound 10 can be converted to either monotosyl 11a or ditosylazide 11 b by treatment with tosyl chloride in the presence of base in a suitable organic solvent. Whether 11a or 11b is produced and to what extent depends on the reaction conditions and stoichiometries of the tosyl chloride reagent. For elaboration to Oseltamivir starting from 11b, the azide functionality on 11b is reduced with a trialkylphosphine preferably such as trimethylphosphine by heating in acetonitrile containing water to produce amine 12. Conversion of amine 12 to aziridine 14 was accomplished in 95% yield by a two step sequence. First, selective bromination of the allylic tosylate group in compound 12 by treatment with a bromide source, such as lithium bromide in ethanol, yielded trans-bromoamine 13. This compound was then cyclized to aziridine 14 by heating in an organic solvent, preferably a chlorinated solvent such as dichloromethane (DCM), in the presence of a trialkylamine base, for example triethylamine, to furnish compound 14. The aziridine ring on compound 14 was then acetylated using an acetylating agent, most preferably acetyl chloride, in the presence of a base, such as triethylamine, to provide acetylaziridine 15. The 3-pentylether side chain was installed by Lewis-acid mediated acetylaziridine ring-opening with 3-pentanol to yield compound 16. A preferred Lewis-acid is boron trifluoride etherate.

An alternative preparation of compound 16 is depicted in FIG. 6. Here the starting point is the 3-O-monotosyl compound 11a which is obtained by mono-tosylation of compound 10. The 5-O-hydroxyl group is protected with, for example, a silylether protecting group. This is followed by similar chemistry as was accomplished to convert compounds 1b to 16 to convert 19 to 16. Thus, 19 was subjected to trialkylphosphine reduction, bromination, aziridine-ring formation, acetylation, and Lewis-acid mediated introduction of the 3-pentyloxy moiety. After this, the protecting group (for instance, a tert-butyldiphenylsilyl as shown in FIG. 6) can be removed and the resulting alcohol tosylated to give compound 16.

Treatment of compound 16, the common intermediate in FIGS. 5 and 6, with sodium azide in a polar solvent such as N,N-dimethylformamide (DMF) afforded azide 17 (FIG. 5) which can be converted to the final drug substance 18 by using reported procedures (for instance, J. C. Rohloff et al., J. Org. Chem., 63, 1998, pp. 4545-4550). Thus, compound 17 was subjected to catalytic hydrogenation with Lindlar's catalyst in ethanol (1 atm H₂) to provide Oseltamivir free base. This was then treated with 85% phosphoric acid (1 eq) to form Oseltamivir phosphate (18).

According to yet another aspect of the invention, there is provided a process for the preparation of Oseltamivir phosphate 18, comprising the steps of:
i) converting compound 17 to Oseltamivir phosphate 18 by azide reduction and salt formation; and
ii) converting compound 16 to compound 17 by azide displacement.

According to yet another aspect of the invention, there is provided a process for the preparation of Oseltamivir phosphate 18, comprising the steps of:
i) converting compound 17 to Oseltamivir phosphate 18 by azide reduction and salt formation;
ii) converting compound 16 to compound 17 by azide displacement; and
iii) converting compound 15 to compound 16 by Lewis-acid mediated aziridine ring opening in the presence of 3-pentanol.

According to yet another aspect of the invention, there is provided a process for the preparation of Oseltamivir phosphate 18, comprising the steps of:
i) converting compound 17 to Oseltamivir phosphate 18 by azide reduction and salt formation;
ii) converting compound 16 to compound 17 by azide displacement;
iii) converting compound 15 to compound 16 by Lewis-acid mediated aziridine ring opening in the presence of 3-pentanol; and
iv) converting compound 14 to compound 15 by acetylation.

According to yet another aspect of the invention, there is provided a process for the preparation of Oseltamivir phosphate 18, comprising the steps of:
i) converting compound 17 to Oseltamivir phosphate 18 by azide reduction and salt formation;
ii) converting compound 16 to compound 17 by azide displacement;
iii) converting compound 15 to compound 16 by Lewis-acid mediated aziridine ring opening in the presence of 3-pentanol;
iv) converting compound 14 to compound 15 by acetylation; and
v) converting compound 13 to compound 14 by base-mediated aziridine ring formation.

According to yet another aspect of the invention, there is provided a process for the preparation of Oseltamivir phosphate 18, comprising the steps of:
i) converting compound 17 to Oseltamivir phosphate 18 by azide reduction and salt formation;
ii) converting compound 16 to compound 17 by azide displacement;
iii) converting compound 15 to compound 16 by Lewis-acid mediated aziridine ring opening in the presence of 3-pentanol;
iv) converting compound 14 to compound 15 by acetylation;
v) converting compound 13 to compound 14 by base-mediated aziridine ring formation; and
vi) converting compound 12 to compound 13 by bromide displacement.

According to yet another aspect of the invention, there is provided a process for the preparation of Oseltamivir phosphate 18, comprising the steps of:
i) converting compound 17 to Oseltamivir phosphate 18 by azide reduction and salt formation;
ii) converting compound 16 to compound 17 by azide displacement;
iii) converting compound 15 to compound 16 by Lewis-acid mediated aziridine ring opening in the presence of 3-pentanol;
iv) converting compound 14 to compound 15 by acetylation;
v) converting compound 13 to compound 14 by base-mediated aziridine ring formation;
vi) converting compound 12 to compound 13 by bromide displacement, and
vii) converting compound 1b to compound 12 by trialkylphosphine mediated azide reduction.

According to yet another aspect of the invention, there is provided a process for the preparation of Oseltamivir phosphate 18, comprising the steps of:
i) converting compound 17 to Oseltamivir phosphate 18 by azide reduction and salt formation;
ii) converting compound 16 to compound 17 by azide displacement;
iii) converting compound 15 to compound 16 by Lewis-acid mediated aziridine ring opening in the presence of 3-pentanol;
iv) converting compound 14 to compound 15 by acetylation;
v) converting compound 12 to compound 13 by bromide displacement;
vi) converting compound 13 to compound 14 by base-mediated aziridine ring formation;
vii) converting compound 1b to compound 12 by trialkylphosphine mediated azide reduction; and
viii) converting compound 10 to compound 11b by ditosylation.

According to yet another aspect of the present invention there is provided a process for the preparation of Oseltamivir Phosphate 18 comprising the steps of:
i) converting compound 17 to Oseltamivir phosphate 18 by azide reduction and salt formation;
ii) converting compound 16 to compound 17 by azide displacement;
iii) converting compound 15 to compound 16 by Lewis-acid mediated aziridine ring opening in the presence of 3-pentanol;
iv) converting compound 14 to compound 15 by acetylation;
v) converting compound 13 to compound 14 by base-mediated aziridine ring formation;
vi) converting compound 12 to compound 13 by bromide displacement;
vii) converting compound 1b to compound 12 by trialkylphosphine mediated azide reduction;
viii) converting compound 10 to compound 11b by ditosylation;
ix) converting compound 9 to compound 10 by base treatment to achieve intramolecular cyclization;
x) converting compound 8 to compound 9 by acid hydrolysis;
xi) converting compound 7 to compound 8 by displacement of the triflate group using the anion of dialkylphosphonoacetate;
xii) converting compound 1 to compound 7 by triflation of the primary hydroxyl; and either
xiii) (a) converting D-glucose to compound 1 by acetonide formation, triflation, azide displacement, acetonide hydrolysis, periodate cleavage, and reduction,
   (b) or converting D-xylose to compound 1 by diacetonide formation, selective acetonide hydrolysis, protection of the primary hydroxyl group, triflation, azide displacement, and removal of the primary hydroxyl protecting group.

According to yet another aspect of the present invention there is provided a process for the preparation of Oseltamivir Phosphate comprising the steps of:
i) converting compound 17 to Oseltamivir phosphate 18 by azide reduction and salt formation;
ii) converting compound 16 to compound 17 by azide displacement;
iii) converting compound 23 to compound 16 by:
   1) a Lewis acid-mediated ring-opening of the acetylaziridine ring of compound 23 in the presence of 3-pentanol;
   2) followed by a deprotecting step using a deprotecting agent; and
   3) followed by a tosylation step;
iv) compound 23 is prepared by acetylating a compound 22 using an acetylating agent in the presence of a base;
v) compound 22 is prepared by treating a compound 21 with a base in a suitable solvent;
vi) compound 21 is prepared by treating a compound 20 with lithium bromide in alcohol;
vii) compound 20 is prepared by reducing a compound 19 using a trialkylphosphine in tetrahydrofuran containing water;
viii) compound 19 is prepared by treating a compound 11a with a silylating reagent;
ix) compound 10 is converted to compound 11a by tosylation;
x) converting compound 9 to compound 10 by base treatment to achieve intramolecular cyclization;
xi) converting compound 8 to compound 9 by acid hydrolysis;
xii) converting compound 7 to compound 8 by displacement of the triflate group using the anion of dialkylphosphonoacetate;
xiii) converting compound 1 to compound 7 by triflation of the primary hydroxyl; and either
xiv) (a) converting D-glucose to compound 1 by acetonide formation, triflation, azide displacement, acetonide hydrolysis, periodate cleavage, and reduction;
   (b) or converting D-xylose to compound 1 by diacetonide formation, selective acetonide hydrolysis, protection of the primary hydroxyl group, triflation, azide displacement, and removal of the primary hydroxyl protecting group.

According to yet another aspect of the present invention there is provided a process for the preparation of Oseltamivir Phosphate 18 comprising the steps of:
i) converting compound 27 to compound 28 to compound 18 by:
   1) Lewis-acid mediated ring opening in the presence of 3-pentanol;
   2) azide displacement;
   3) azide reduction; and
   4) salt formation;
ii) converting a compound 26 to a compound 27 by:
   1) base mediated aziridine ring formation, and
   2) acetylation using an acetylating agent,
iii) treating a compound 25 to a bromide displacement step to yield compound 26;
iv) treating a compound 24 to an azide reduction step to yield compound 25;
v) converting a compound 6 to a compound 24 by protection of the alcoholic groups;
vi) converting compound 5 to compound 6 by base treatment to achieve intramolecular cyclization;
vii) converting compound 4 to compound 5 by acid hydrolysis;
viii) converting compound 2 to compound 4 by displacement of the triflate group using the anion of dialkylphosphonoacetate;
ix) converting compound 1 to compound 2 by triflation of the primary hydroxyl; and either
x) (a) converting D-glucose to compound 1 by acetonide formation, triflation, azide displacement, acetonide hydrolysis, periodate cleavage, and reduction;
   (b) or converting D-xylose to compound 1 by diacetonide formation, selective acetonide hydrolysis, protection of the primary hydroxyl group, triflation, azide displacement, and removal of the primary hydroxyl protecting group.

In a general sense, the synthesis of 18 may be accomplished according to FIG. 7 wherein R′ is selected from substituted or unsubstituted, linear or branched alkyl, aryl, or aralkyl group. Preferably, R′ is selected from a substituted or unsubstituted C1 to C6 alkyl group, a substituted or unsubstituted C6 to C9 aryl group, or a substituted or unsubstituted C7 to C10 aralkyl group. Most preferably, R′ is ethyl, and R″ is a leaving group selected from mesyloxy, trifloxy or tosyloxy. Most preferably, R″ is tosyloxy. R′″ is a leaving group the same as defined above for R″, or a protecting group most preferably selected from silyloxy groups. Most preferably, R′″ is tosyloxy.

Another object of the invention provides for the following novel useful intermediates: 3-azido-3-deoxy-1,2-O-isopropylidene-5-O-trifluoromethanesulfonyl-α-D-ribofuranose (having the structure defined by compound 7); ethyl (3-azido-3-deoxy-5,6-dideoxy-6R/S-diethoxyphosphoryl-1,2-O-isopropylidene-α-D-ribo-heptofuranose)uronate (having the structure defined by compound 8); ethyl (3-azido-3-deoxy-5,6-dideoxy-6R/S-diethoxyphosphoryl-α/β-D-ribo-heptofuranose)uronate (having the structure defined by compound 9); ethyl (3S,4R,5R)-4-azido-3,5-dihydroxycyclohex-1-ene-carboxylate (having the structure defined by compound 10); ethyl (3S,4R,5R)-4-azido-5-hydroxy-3-tosyloxycyclohex-1-ene-carboxylate (having the structure defined by compound 11a); ethyl (3S,4R,5R)-4-azido-3,5-ditosyloxycyclohex-1-ene-carboxylate (having the structure defined by compound 11b); ethyl (3S,4R,5R)-4-amino-3,5-ditosyloxycyclohex-1-ene-carboxylate (having the structure defined by compound 12); ethyl (3R,4R,5R)-4-amino-3-bromo-5-tosyloxycyclohex-1-ene-carboxylate (having the structure defined by compound 13); ethyl (3S,4R,5R)-3,4-imino-5-tosyloxycyclohex-1-ene-carboxylate (having the structure defined by compound 14); ethyl (3S,4R,5R)-3,4-acetylimino-5-tosyloxycyclohex-1-ene-carboxylate (having the structure defined by compound 15); ethyl (3R,4R,5R)-4-acetamido-3-(3-pentyloxy)-5-tosyloxycyclohex-1-ene-carboxylate (having the structure defined by compound 16); ethyl (3S,4R,5R)-4-azido-5-(tert.butyldiphenyl)silyloxy-3-tosyloxycyclohex-1-ene-carboxylate (having the structure defined by compound 19); ethyl (3S,4R,5R)-4-amino-5-(tert.butyldiphenyl)silyloxy-3-tosyloxycyclohex-1-ene-carboxylate (having the structure defined by compound 20); ethyl (3S,4R,5R)-3,4-imino-5-(tert.butyldiphenyl)silyloxycyclohex-1-ene-carboxylate (having the structure defined by compound 22); and ethyl (3S,4R,5R)-3,4-acetylimino-5-(tert.butyldiphenyl)silyloxycyclohex-1-ene-carboxylate (having the structure defined by compound 23).

Another object of the invention provides for the compounds of general formula 4:

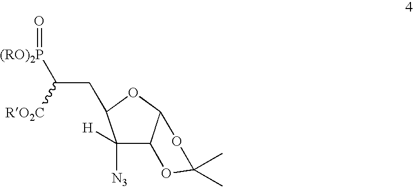

wherein R and R′ are independently selected from substituted or unsubstituted, linear or branched alkyl, aryl, and aralkyl. Preferably, R and R′ are independently selected from substituted or unsubstituted C1 to C6 alkyl. Also preferably, R and R′ are independently selected from substituted or unsubstituted C6 to C9 aryl.

Also preferably, R and R′ are independently selected from substituted or unsubstituted C7 to C10 aralkyl. More preferably, R and R′ are both ethyl.

Another object of the invention provides for the compounds of general formula 5:

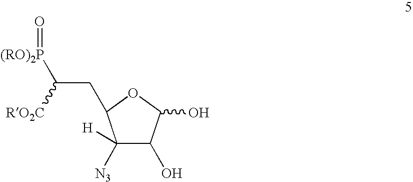

wherein R and R′ are independently selected from substituted or unsubstituted, linear or branched alkyl, aryl, and aralkyl. Preferably, R and R′ are independently selected from substituted or unsubstituted C1 to C6 alkyl. Also preferably, R and R′ are independently selected from substituted or unsubstituted C6 to C9 aryl. Also preferably, R and R′ are independently selected from substituted or unsubstituted C7 to C10 aralkyl. More preferably, R and R′ are both ethyl.

Another object of the invention provides for the compounds of general formula 6:

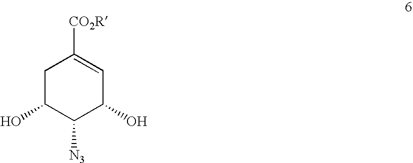

wherein R′ is selected from substituted or unsubstituted, linear or branched alkyl, aryl, and aralkyl. Preferably, R′ is selected from substituted or unsubstituted C1 to C6 alkyl. Also preferably, R′ is selected from substituted or unsubstituted C6 to C9 aryl. Also preferably, R′ is selected from substituted or unsubstituted C7 to C10 aralkyl. More preferably, R′ is ethyl.

Another object of the invention provides for the compounds of general formula 24:

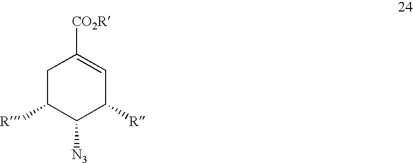

wherein R′ is selected from substituted or unsubstituted, linear or branched alkyl, aryl, or aralkyl group. Preferably, R′ is selected from a substituted or unsubstituted C1 to C6 alkyl group, a substituted or unsubstituted C6 to C9 aryl group, or a substituted or unsubstituted C7 to C10 aralkyl group. Most preferably, R′ is ethyl. R″ is a leaving group selected from mesyloxy, trifloxy or tosyloxy. Most preferably, R″ is tosyloxy. R′″ is a leaving group the same as defined above for R″, or a protecting group most preferably selected from silyloxy groups. Most preferably, R′″ is tosyloxy.

Another object of the invention provides for the compounds of general formula 25:

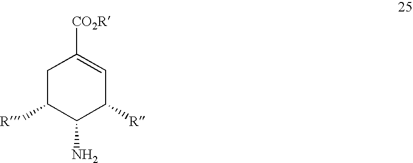

wherein R′ is selected from substituted or unsubstituted, linear or branched alkyl, aryl, or aralkyl group. Preferably, R′ is selected from a substituted or unsubstituted C1 to C6 alkyl, substituted or unsubstituted C6 to C9 aryl, or a substituted or unsubstituted C7 to C10 aralkyl. Most preferably, R′ is ethyl. R″ is a leaving group selected from mesyloxy, trifloxy or tosyloxy. Most preferably, R″ is tosyloxy. R′″ is a leaving group the same as defined above for R″, or a protecting group most preferably selected from silyloxy groups. Most preferably, R′″ is tosyloxy.

Another object of the invention provides for the compounds of general formula 26:

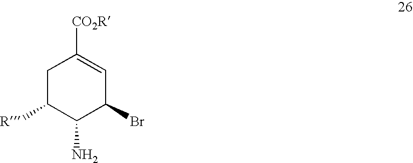

wherein R′ is selected from substituted or unsubstituted, linear or branched alkyl, aryl, or aralkyl group. Preferably, R′ is selected from a substituted or unsubstituted C1 to C6 alkyl group, a substituted or unsubstituted C6 to C9 aryl group, or a substituted or unsubstituted C7 to C10 aralkyl group. Most preferably, R′ is ethyl. R′″ is a leaving group selected from mesyloxy, trifloxy or tosyloxy, or a protecting group most preferably selected from silyloxy groups. Most preferably, R′″ is tosyloxy.

Another object of the invention provides for the compounds of general formula 27:

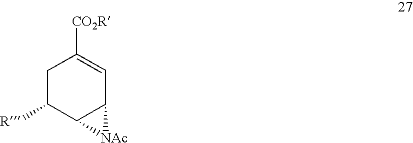

wherein R′ is selected from substituted or unsubstituted, linear or branched alkyl, aryl, and aralkyl. Preferably, R′ is selected from a substituted or unsubstituted C1 to C6 alkyl group, a substituted or unsubstituted C6 to C9 aryl group, or a substituted or unsubstituted C7 to C10 aralkyl group. Most preferably, R′ is ethyl. R′″ is a leaving group selected from mesyloxy, trifloxy or tosyloxy, or a protecting group most preferably selected from silyloxy groups. Most preferably, R′″ is tosyloxy.

Another object of the invention provides for the compounds of general formula 28:

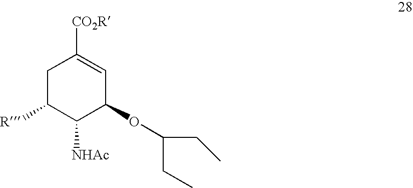

wherein R′ is selected from substituted or unsubstituted, linear or branched alkyl, aryl, and aralkyl. Preferably, R′ is selected from a substituted or unsubstituted C1 to C6 alkyl, a substituted or unsubstituted C6 to C9 aryl, or a substituted or unsubstituted C7 to C10 aralkyl. Most preferably, R′ is ethyl. R′″ is a leaving group selected from mesyloxy, trifloxy or tosyloxy, or a protecting group most preferably selected from silyloxy groups. Most preferably, R′″ is tosyloxy.

### BRIEF DESCRIPTION OF THE FIGURES

FIG. 1 is a chemical depiction of Oseltamivir Phosphate.

FIG. 2 is a schematic showing the stereochemistry of Oseltamivir, an intermediate and the starting reagents of the present invention.

FIG. 3 is a scheme depicting the steps of an embodiment of the process according to the present invention which lead to the formation of compound 6.

FIG. 4 is a scheme depicting the steps of an embodiment of the process according to the present invention which lead to the formation of Compound 10.

FIG. 5 is a scheme depicting the steps of an embodiment of the process according to the present invention starting from compound 10 and which lead to the formation of Compound 18 (Oseltamivir Phosphate).

FIG. 6 is a scheme depicting the steps of an embodiment of the process according to the invention for the formation of Compound 18 (Oseltamivir Phosphate) starting from the intermediate compound 11a.

FIG. 7 is a scheme depicting the steps of an embodiment of the process according to the present invention for the formation of compound 18 (Oseltamivir Phosphate) starting from the intermediate compound 6.

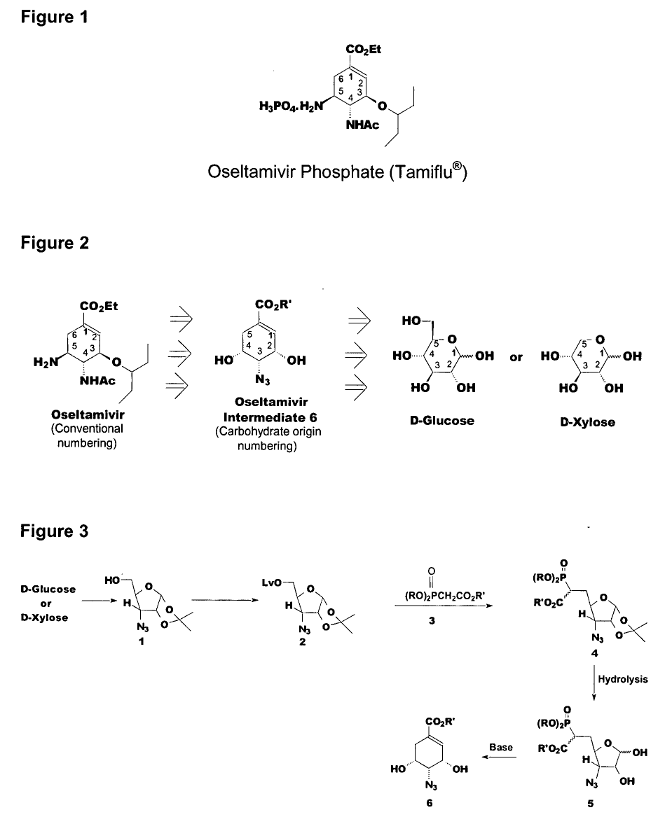

### DESCRIPTION OF A PREFERRED EMBODIMENT

Preferably, the reaction detailed in FIG. 3 is carried out according to the following: the conversion of D-glucose or D-xylose to compound 1 by processes known in the prior art such as R. L. Whistler et al., J. Org. Chem., (1972) 37, 3187 and P. Stazewski et al., Tetrahedron (1998) 54, 13529. Subsequently, the primary hydroxyl on compound 1 is converted into a leaving group, for instance a sulfonate ester, most preferably a triflate. This is accomplished, for instance, by contacting compound 1 with the corresponding sulfonyl chloride or sulfonic anhydride in the presence of a base. The leaving group is then displaced using, for example, a phosphonoacetate ester 3 and a suitable inorganic base, most preferably sodium hydride to form compound 4. The structure of compound 3 may be:

(RO)₂P(O)CH₂CO₂R′ 3

wherein R and R′ are independently selected from substituted or unsubstituted linear or branched C1 to C6 alkyl, C6 to C9 aryl, and C7 to C10 aralkyl, most preferably R and R′ are both ethyl. The acetonide functionality on the phosphonoacetate ester 4 may then be removed by hydrolysis to provide the diol 5 which may then be cyclized to the cyclohexene derivative 6 by base treatment.

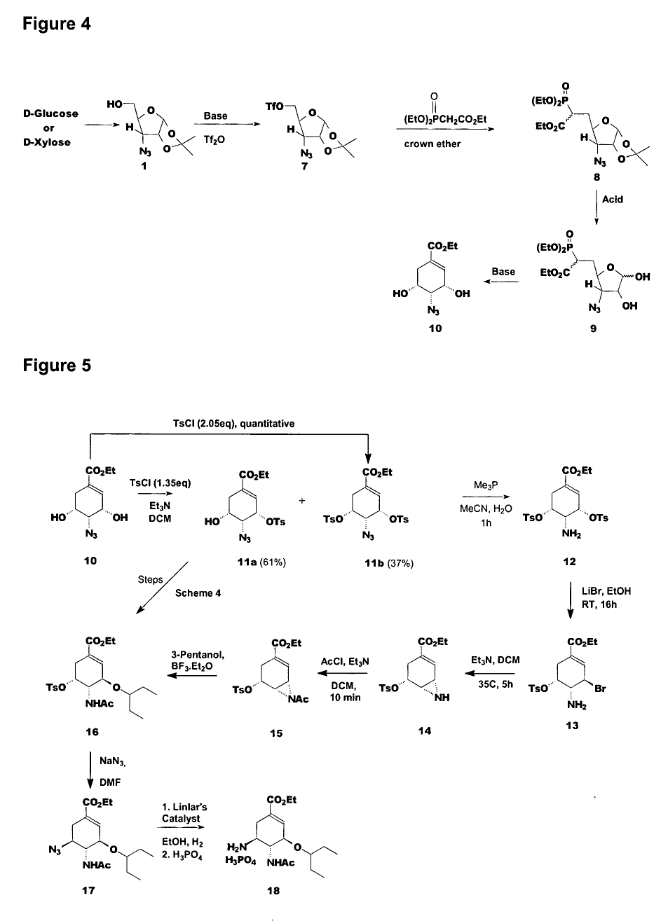

Preferably, the reaction detailed in FIG. 4 is carried out according to the following: Compound 1 is converted to triflate 7 which is believed to be a new compound. Triflate 7 is depicted with the correct D-ribo-configuration (see U.S. Pat. No. 6,020,344) but it is clear from the text of the patent that the depiction is a graphical error and it should have been depicted with the D-xylo-configuration according to U.S. Pat. No. 6,020,344. Compound 7 is prepared from compound 1 by, for instance, the dropwise addition of triflic anhydride in a suitable solvent, for example dichloromethane, to a solution of compound 1 in a suitable solvent, such as dichloromethane, containing a base. Suitable bases include trialkylamines, for example triethylamine. Suitable temperatures for the reactions range from about −40° C. to about 20° C., more preferably ranging from about −30° C. to about −10° C., most preferably about −20° C. After the reaction is completed, it is quenched using, for instance, aqueous sodium bicarbonate and compound 7 is obtained. Compound 7 is isolated using standard processing techniques. A solution of triethyl phosphonoacetate, a suitable inorganic base, most preferably sodium hydride, and a crown ether, most preferably 15-crown-5 in a suitable solvent, for instance N,N-dimethylformamide is prepared and added slowly to a solution of compound 7 in N,N-dimethylformamide at room temperature. After the reaction is completed, it was quenched with a suitable quenching agent, for example 1M potassium dihydrogenphosphate, and compound 8 was isolated after work-up. Compound 8 is hydrolyzed to compound 9 using an organic or inorganic acid, most preferably a solution containing about 60% aqueous trifluoroacetic acid at temperatures ranging from about 10° C. to about 50° C., most preferably from about 25° C. to about 30° C. for a suitable length of time. The reaction is processed using, for example, liquid-liquid extractive techniques, and purified. Compound 9 is cyclized by adding a base, for instance sodium hydride, to yield a solution of compound 10 in an anhydrous solvent, for instance tetrahydrofuran at temperatures ranging from about −30° C. to about 20° C., more preferably ranging from about −20° C. to about 10° C., most preferably at about 0° C. After reaction completion, it was worked up and purified to yield cyclohexene derivative 10.

Compound 10 (or the corresponding general compound 6) can be further elaborated to Oseltamivir by first forming the ditosyl compound with tosylchloride (2.05 eq) in a suitable solvent such as dichloromethane or toluene in the presence of triethylamine to provide 11b (FIG. 5). The C3 azide moiety of compound 11b is then converted to an amino functionality by reduction with a trialkylphosphine such as trimethylphosphine in acetonitrile containing some water. Cyclization of amine 12 to acetylaziridine 15 is accomplished by a three step sequence. First, the allylic tosylate group of compound 12 is selectively displaced with bromide ion by the treatment with a brominating agent such as lithium bromide in ethanol to yield trans-bromoamine 13. Second, the cyclization of bromoamine 13 to aziridine 14 is accomplished by heating in an organic solvent, preferably dichloromethane (DCM) in the presence of a base, for example triethylamine. Finally, the acetylation of an aziridine such as 14 is accomplished using an acetylating agent such as acetyl chloride and a base, such as triethylamine, to yield acetylaziridine 15. The 3-pentylether side chain is introduced by Lewis acid-mediated ring-opening of the acetylaziridine ring of compound 15 with 3-pentanol to provide compound 16. The preferred Lewis acid is boron trifluoride diethyl etherate. The compound 16 is treated with an azide source, most preferably sodium azide in a suitable solvent, most preferably dimethylformamide (DMF). This afforded the known azide 17 which is converted to the final drug substance 18 using known procedures (for instance, J. C. Rohloff et al., J. Org. Chem., 63, 1998, pp. 4545-4550). This involved catalytic hydrogenation with Lindlar's catalyst in ethanol (1 atm H₂) followed by the treatment of the resulting free base with 85% phosphoric acid (1 eq).

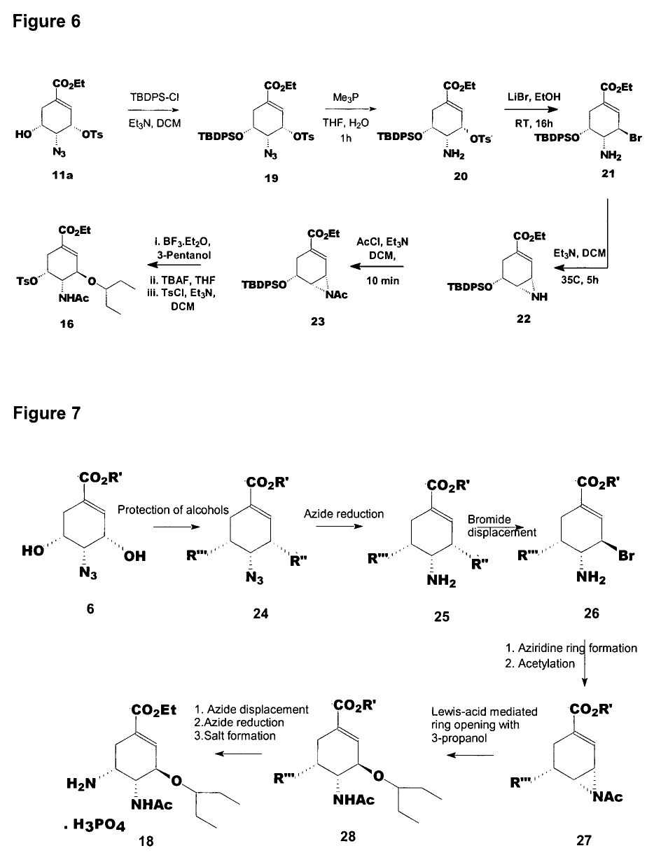

In an alternative route (FIG. 6), Compound 10 (or the corresponding general compound 6) is transformed into Oseltamivir by first forming the 3-monotosyl compound 11a with tosylchloride (1.35 eq) in a suitable solvent such as dichloromethane or toluene in the presence of a base such as triethylamine. The 5-hydroxyl moiety of 11a was protected, most preferably as its silylether, most preferably the tert-butyldiphenylsilyl (TBDMS) ether using tert-butyldiphenylsilyl chloride as the silylating reagent. This provided 19 in 98% yield. The next step involved the reduction of azide 19 to the amine 20 using a trialkylphosphine, most preferably trimethylphosphine, in tetrahydrofuran containing some water. The treatment of compound 20 with lithium bromide in an alcohol such as ethanol or 3-pentanol provided the bromoamine 21, which was converted to aziridine 22 by treatment with a base such as triethylamine in a suitable solvent. Examples of the suitable solvent include dichloromethane. The aziridine 22 was then acetylated using an acetylating agent such as acetyl chloride in presence of a base, such as triethylamine, to obtain compound 23. Compound 23 was subsequently converted to compound 16 by Lewis acid-mediated ring-opening of the acetylaziridine ring using 3-pentanol. This was followed by deprotection using a deprotecting agent, for instance tert-butylammonium fluoride, and tosylation to provide 16 which could be further converted to Oseltamivir as described previously.

Furthermore, if desired, a person skilled in the art would know that one could convert the ester functionality on compound 10 (or general compound 6), or the precursor intermediates used to prepare compounds 10 and 6, to the corresponding carboxylic acid by known processes such as hydrolysis or hydrogenolysis where R′ is hydrogen.

The following examples are merely representative of the present invention and are not intended to be limiting.

### EXAMPLE 1

### 3-Azido-3-deoxy-1,2-di-O-isopropylidene-α-D-ribofuranoside 1

3-Azido-3-deoxy-1,2-di-O-isopropylidene-α-D-ribofuranoside (1) was prepared from glucose following procedures based on those described in R. L. Whistler et al., J. Org. Chem., (1972) 37, 3187 and P. Stazewski et al., Tetrahedron (1998) 54. To a mixture of glucose (220 g, 1099 mmol) in acetone (3.6 L) was added iodine (14.1 g, 55.4 mmol) and acetic anhydride (170 g, 1667 mmol) at room temperature. The mixture was refluxed at 59° C. for 3 h and allowed to cool whereupon triethylamine (338 g) was added slowly at ambient temperature, filtered the solid and washed twice with acetone (100 mL). The filtrate was concentrated under vacuum and water was added (600 mL). The organic layer was extracted thrice with toluene (600 mL) and the combined organic phases were concentrated. Heptane (800 mL) was added with stirring, filtered and the solid, washed with heptane-acetone (2:1, 750 mL) to obtain white crystalline solid (217 g, 75%) 1, 2:5,6-di-O-isopropylidene-α-D-glucofuranoside, or diacetone glucose.

The above diacetone glucose (200 g, 786 mmol) was dissolved in dichloromethane (2.7 L) and pyridine (121 g, 1.53 mmol) was added. The mixture was cooled to −10° C. and trifluoromethanesulfonic anhydride (257 g, 911 mmol) was added dropwise and stirred for 1 hour at −10° C. Water (1.6 L) was added to the mixture and allowed to warm to ambient temperature and the organic phase was separated. The aqueous layer was extracted twice with 300 mL dichloromethane and the combined organic phases were washed twice with 450 mL water and evaporated in vacuo at a temperature below 35° C. The residue was taken up in diethyl ether (1 L) and extracted with cold 2 N hydrochloric acid (1 L). The organic phase was separated and the aqueous layer was extracted with diethyl ether (100 mL) and the combined organic phases were washed with water, brine (400 mL each) and saturated aqueous sodium bicarbonate solution (100 mL). The organic phase was filtered through 250 g silica gel and the silica gel was eluted with 2 L diethyl ether. Evaporation of the solution and drying under high vacuum afforded 285.0 g (94.5%) of 1, 2:5,6-Di-O-isopropylidene-3-O-trifluoromethanesulfonyl-α-D-glucofuranoside.

To dimethylformamide (1.1 L), were added sodium azide (48.1 g, 740 mmol) and tetrabutylammonium chloride hydrate (0.40 g, ca. 1.4 mmol). The mixture was heated to 50-55° C. and a solution of diacetone-D-glucose triflate (145.0 g, 370 mmol) in dimethylformamide (335 mL) was added over 2 hours. After complete addition, the mixture was stirred at 50° C. for 2 h and cooled to ambient temperature. Water (1.9 L) was added and the pH was adjusted to 7.8 by addition of solid sodium bicarbonate. Toluene (750 mL) was added and organic phase was separated. The lower aqueous was extracted twice with 750 mL toluene and the combined organic phases were washed twice with water and brine (500 mL each), filtered through anhydrous sodium sulfate and evaporated to 100.2 g of a yellow oil composed of composed of product and the elimination product 3-deoxy-3,4-didehydro-1,2:5,6-di-O-isopropylidene-α-D-allofuranose.

Water (360 mL) and glacial acetic acid (1.1 L) were added to the mixture (107.3 g; ca. 403 mmol) and the mixture was heated at 50° C. for 3 hours and evaporated in vacuo. Toluene (200 mL) was added and concentrated to remove traces of acetic acid. The crude diol was dissolved in 2 L ethanol at 0° C. and sodium metaperiodate (130 g, 607 mmol) in 1 L water was added at 10° C. and stirred for 2.5 h. Sodium borohydride (30.50 g, 606 mmol) was then added in portions. The mixture was stirred overnight and allowed to warm to ambient temperature. The mixture was filtered and washed with 200 mL ethanol. The filtrate was evaporated to dryness and the residue was taken up in 1.5 L ethyl acetate, extracted with saturated aqueous sodium bicarbonate solution, water (500 mL each), and brine (200 mL). The organic phase was dried over anhydrous sodium sulfate, filtered and evaporated to yield 25.03 g of yellow oily compound. The crude was filtered through 350 g silica gel and eluted with 2 L ethyl acetate/hexanes 1:1 to give 20.62 g (48%) of 3-azido-3-deoxy-1,2-di-O-isopropylidene-α-D-ribofuranoside (1).

### EXAMPLE 2

### 3-azido-3-deoxy-1,2-O-isopropylidene-5-O-trifluoromethanesulfonyl-α-D-ribofuranose 7

Compound 1 (1.16 g, 5.40 mmol) was dissolved in dichloromethane. Triethyl amine (0.8 mL, 9.8 mmol) was added and cooled to −20° C. Triflic anhydride (1.68 g, 5.94 mmol) dissolved in dichloromethane was added. The reaction was quenched with saturated aqueous sodium bicarbonate, diluted with ethyl acetate and the phases were separated. The aqueous phase was extracted with ethyl acetate and the combined organic phases were dried with anhydrous sodium sulfate, concentrated, passed through a short silica gel column using ethyl acetate/hexanes as the eluant and concentrated to give 7 as a light yellow oil (1.26 g, 3.63 mmol, 67.2%).

¹H NMR (300 MHz-CDCl₃) δ1.39 (s, 3H, CH₃), 1.59 (s, 3H, CH₃), 3.53 (dd, 1H, H-3), 4.23 (dt, 1H, H-4), 4.59 (dd, 1H, H-5a), 4.80-4.85 (m, 2H, H-2, H-5b), 5.86 (d, J1, 2=3.53, 1H, H-1), ppm.

### EXAMPLE 3

### Ethyl (3-Azido-3-deoxy-5,6-dideoxy-6R/S-diethoxyphosphoryl-1,2-O-isopropylidene-α-D-ribo-heptofuranose)uronate 8

Triethyl phosphonoacetate (3.11 g, 13.84 mmol) was added to a mixture of 60% sodium hydride (507 mg, 12.68 mmol) and 15-crown-5 (15 μL) in N,N-dimethylformamide and stirred at room temperature, for 1 hour. A solution of compound 7 (4.0 g, 11.53 mmol) in N,N-dimethylformamide was added slowly over a period of 15 minutes. After the reaction was complete, it was quenched with 1M potassium dihydrogen phosphate, diluted with ether and the phases were separated. The aqueous phase was extracted with ether and the combined organic phases were concentrated and purified using silica gel column using acetate/hexanes as the eluant to give compound 1 (880 mg, 4.13 mmol, 35.8%) and compound 8 (2.55 g, 6.06 mmol, 52.5%).

¹H NMR (300 MHz-CDCl₃) δ 1.27-1.37 (m, 12H, CH₃, 3×CH₂CH₃), 1.53 (s, 3H, CH₃), 2.01 (m, 0.5H, H-5Aa), 2.29 (m, 1H, H-5Ba, H-5Bb), 2.52 (m, 0.5H, H-5Ab), 3.05 (m, 1H, H-3), 3.24 (m, 1H, H-6), 4.04 (m, 0.5H, H-4A), 4.19 (m, 6.5H, H-4B, 3×CH₂CH₃), 4.73 (bs, H, H-2), 5.77 (bs, 1H, H-1), ppm.

### EXAMPLE 4

### Ethyl (3-Azido-3-deoxy-5,6-dideoxy-6R/S-diethoxyphosphoryl-α/β-D-ribo-heptofuranose)uronate 9

Compound 8 (2 g, 4.75 mmol) was dissolved in 60% aqueous trifluoroacetic acid (8 mL) at room temperature and then the temperature was increased to 30° C. When the reaction was complete, it was diluted with toluene and the solution was concentrated and the residue was purified by silica column chromatography using ethyl acetate/hexanes as the eluant to give compound 9 (1.74 g, 4.57 mmol, 96.1%).

¹H NMR (300 MHz-CDCl₃) δ 1.24-1.37 (m, 9H, 3×CH₃), 1.53 (s, 3H, CH₃), 1.86-2.55 (m, 2H, H-5Aa, H-5Ab, H-5Ba, H-5Bb), 3.00-3.40 (m, 2H, H-3, H-6), 3.58 (m, 1H, H-2), 4.05 (m, 1H, H-4), 4.11-4.34 (m, 3×CH₂CH₃), 5.29-5.34 (m, 1H, H-1), ppm.

### EXAMPLE 5

### Ethyl (3S,4R,5R)-4-Azido-3,5-dihydroxycyclohex-1-enecarboxylate 10

Compound 9 (690 mg, 1.81 mmol) was dissolved in tetrahydrofuran, cooled to 0° C. and 60% sodium hydride (127 mg, 3.17 mmol) was added portionwise. After the reaction was complete, the mixture was cooled to 0° C. and neutralized with 1M KH₂PO₄, diluted with ethyl acetate and the phases were separated. The aqueous phase was extracted with ethyl acetate and the combined organic phases were washed with brine, dried with sodium sulfate, concentrated and purified by silica gel chromatography using ethyl acetate/hexanes to give 270 mg (1.19 mmol, 65.7%) of the compound 10.

¹H NMR (300 MHz-CDCl₃) δ 1.31(t, 3H, CH₂CH₃), 2.47(bs, 1H, exchangeable, C5-OH), 2.55-2.63(m, 2H, ring CH₂), 2.76(bd, 1H, exchangeable, C5-OH), 3.90(m, 1H, H-5), 4.19-4.26(m, 3H, CH₂CH₃, H-4), 4.49(bs, 1H, H-3), 6.82(s, 1H, H-2) ppm.

### EXAMPLE 6

### Ethyl (3S,4R,5R)-4-azido-5-hydroxy-3-tosyloxycyclohex-1-ene-carboxylate 11a and Ethyl (3S,4R,5R)-4-azido-3,5-ditosyloxycyclohex-1-ene-carboxylate 11b

Triethylamine (0.34 ml, 2.40 mmol) was added to a solution of compound 10 (0.32 g, 1.41 mmol) in dichloromethane (8 mL) at −20° C. followed by the addition of tosylchloride (0.27 g, 1.41 mmol) in dichloromethane (2 mL) over a period of 0.5 h under a nitrogen atmosphere. The mixture was allowed to warm to room temperature and maintained for 16 h. The reaction mixture was cooled to −20° C., and tosylchloride (0.05 g, 0.28 mmol) in dichloromethane (1 mL) was added and stirring continued for 3 h while allowing to warm to room temperature. After reaction completion was confirmed by TLC, 5% aq NaHCO₃ (5 mL) was added and the phases were separated. The aqueous layer was extracted with dichloromethane (5 mL) and the combined organic layers were concentrated to dryness and the residue was purified by silica gel column chromatography using ethyl acetate/heptane (1:4) to afford monotosylate 11a (0.33 g, 61%) and ditosylate 11b (0.28 g, 37%).

Triethylamine (0.2 ml, 1.41 mmol) was added to a solution of compound 10 (0.1 g, 0.44 mmol) in dichloromethane (2 mL) at RT followed by the addition of tosylchloride (0.172 g, 0.903 mmol) under a nitrogen atmosphere. The mixture was stirred for 19 h or till reaction completion was confirmed by TLC. 5% Aq NaHCO₃ (5 mL) was added and the phases were separated and the aqueous layer was extracted with dichloromethane (5 mL) and the combined organic layers were concentrated to dryness to afford ditosylate 11b (0.23 g, 98%).

Compound 11a

¹H NMR (300 MHz-CDCl₃) δ 1.28(t, 3H, CH₂CH₃), 2.12(d, 1H, OH), 2.31(m, 1H, ring CH₂), 2.47(s, 3H, Ts-CH₃), 2.69(dd, 1H, ring CH₂), 3.92(m, 1H, H-5), 4.08(bs, 1H, H-4), 4.19(q, 2H, CH₂CH₃), 5.37(bs, 1H, H-3), 6.52(s, 1H, H-2), 7.38-7.42(d, 2H, Ts), 7.84-7.89(d, 2H, Ts) ppm.

Compound 11b

¹H NMR (300 MHz-CDCl₃) δ 1.25(t, 3H, CH₂CH₃), 2.47-2.58(m, 8H, ring CH₂, Ts-CH₃, Ts-CH₃), 4.12-4.17(m, 3H, CH₂CH₃, H-4), 4.65(m, 1H, H-5), 5.31(bs, 1H, H-3), 6.47(s, 1H, H-2), 7.37-7.42(m, 4H, Ts), 7.79-7.86(m, 4H, Ts) ppm.

### EXAMPLE 7

### Ethyl (3S,4R,5R)-4-amino-3,5-ditosyloxycyclohex-1-ene-carboxylate 12

1M Trimethylphosphine in toluene (2.3 mL, 2.26 mmol) was added to compound 11b (1.1 g, 2.06 mmol) in a mixture of acetonitrile (10 mL) and water (0.5 mL) at room temperature, stirred for 1 h and heated at 45° C. for 45 min. After confirming the formation of ylide by ¹H NMR, the mixture was concentrated to dryness. Ethyl acetate/water (6:1 v/v, 7 mL total) was added, the mixture was heated at 50° C. for 3 h and concentrated to dryness. The residue was purified by silica gel column chromatography using ethyl acetate/heptane (1:3) to afford light yellow sticky ditosylate 12 (0.60 g, 57%).

¹H NMR (300 MHz-CDCl₃) δ 1.21(t, 3H, CH₂CH₃), 2.41-2.78(m, 8H, ring CH₂, Ts-CH₃, Ts-CH₃), 3.52(bs, 1H, H-4), 4.08(q, 2H, CH₂CH₃), 4.66(t, 1H, H-5), 5.14(bs, 1H, H-3), 6.47(s, 1H, H-2), 7.31-7.42(m, 4H, Ts), 7.78-7.89(m, 4H, Ts) ppm.

### EXAMPLE 8

### Ethyl (3R,4R,5R)-4-amino-3-bromo-5-tosyloxycyclohex-1-ene-carboxylate 13

Lithium bromide (0.47 g, 5.41 mmol) was added to a solution of 12 (0.55 g, 1.08 mmol) in ethanol (10 mL) at 0° C. and the solution was allowed to warm to room temperature and maintained for 16 h. The mixture was concentrated to dryness, diluted with dichloromethane (10 mL) and 5% aqueous sodium bicarbonate (2 mL) was added. The phases were separated and the organic phases was evaporated in vacuo to dryness to afford crude bromo compound 13 (0.38 g, 84%).

¹H NMR (300 MHz-CDCl₃) δ 1.22(t, 3H, CH₂CH₃), 2.46(s, 3H, Ts-CH₃), 2.72(m, 2H, ringCH₂), 3.34(m, 1H, H-4), 4.19(m, 2H, CH₂CH₃), 4.55(bs, 1H, H-5), 5.02(m, 1H, H-3), 6.91(bs, 1H, H-2), 7.38(d, 2H, Ts), 7.83(d, 2H, Ts) ppm.

### EXAMPLE 9

### Ethyl (3S,4R,5R)-3,4-imino-5-tosyloxycyclohex-1-ene-carboxylate 14

Triethylamine (0.75 mL, 5.41 mmol) was added to a solution of 13 (0.38 g, 0.91 mmol) in dichloromethane (4 mL) at room temperature and then the solution was heated at 35° C. for 5 h and concentrated to dryness to afford the crude aziridine 14 along with triethylammonium bromide salt (0.53 g, 95%).

¹H NMR (300 MHz-CDCl₃) δ 1.23(t, 3H, CH₂CH₃), 2.21-2.39(m, 1H, 4H), 2.47(s, 3H, Ts-CH₃), 2.61-2.89(m, 3H, H-3, ring CH₂), 4.09(m, 3H, CH₂CH₃, H-5), 4.89(bs, 1H, NH), 7.03(bs, 1H, H-2), 7.37(d, 2H, Ts), 7.82(d, 2H, Ts) ppm.

### EXAMPLE 10

### Ethyl (3S,4R,5R)-3,4-acetylimino-5-tosyloxycyclohex-1-ene-carboxylate 15

Triethylamine (0.015 mL, 0.1 mmol) was added to a mixture of 14 (0.02 g, 0.06 mmol) in dichloromethane (1 mL) at 0° C. Acetyl chloride (0.05 mL, 0.06 mmol) was then added and the mixture allowed to warm to room temperature and stirred for 15 min. Water (1 mL) was added, phases were separated, the organic phase was evaporated in vacuo to dryness and the residue was purified by silica gel column chromatography to afford 15 (0.012 g, 53%).

¹H NMR (300 MHz-CDCl₃) δ 1.28(t, 3H, CH₂CH₃), 2.19(s, 3H, COCH₃), 2.24-2.39(m, 1H, H-4), 2.49(s, 3H, Ts-CH₃), 2.79-2.89(dd, 1H, H-3), 3.22(m, 2H, ring CH₂), 4.19(q, 2H, CH₂CH₃), 4.79(m, 1H, H-5), 7.04(t, 1H, H-2), 7.38(d, 2H, Ts), 7.88(d, 2H, Ts) ppm.

¹³C NMR (300 MHz-CDCl₃) δ 14.09(CH₂CH₃), 21.65(COCH₃), 23.14(Ts-CH₃), 26.32(C-6), 34.95(C-4), 40.32(C-3), 61.14(CH₂CH₃), 75.99(C-5), 127.79(C, C, Ts), 130.09(C, C, Ts), 130.85(C, Ts), 132.72(C, Ts), 133.37(C-1), 145.35(C-2), 164.91 (COCH₂CH₃), 181.57(COCH₃) ppm.

ESI+ 402.24(M+Na).

### EXAMPLE 11

### Ethyl (3R,4R,5R)-4-acetamido-3-(3-pentyloxy)-5-tosyloxycyclohex-1-ene-carboxylate 16

Boron trifluoride diethyl etherate (0.04 mL, 0.32 mmol) was added to a mixture of 15 (0.08 g, 0.21 mmol) in 3-pentanol (2 mL) at 0° C. over a period of 1 hour and allowed to warm to room temperature. Ethyl acetate (3 mL) and 5% aqueous sodium bicarbonate were added, the phases were separated, and the organic phase was concentrated in vacuo to dryness to afford 16 as light yellow sticky solid (0.09 g, 93%).

¹H NMR (300 MHz-CDCl₃), δ 0.88(m, 6H, CHCH₂CH₃), 1.27(t, 3H, CH₂CH₃), 1.49 (m, 4H, CHCH₂CH₃), 1.89(s, 3H, COCH₃), 2.41(m, 5H, Ts-CH₃, ring CH₂), 3.34(m, 1H, H-4), 4.08(m, 1H, CH₂CHCH₂), 4.17(m, 3H, CH₂CH₃, H-3), 4.94(m, 1H, H-5), 5.62-5.65(bd, 1H, NH), 6.83(s, 1H, H-2), 7.36(d, 2H, Ts), 7.83(d, 2H, Ts) ppm.

¹³C NMR (300 MHz-CDCl₃) δ 9.79(CH₃), 14.57(CH₃), 22.07(CH₂), 23.62(CH₂), 26.30(COCH₃), 26.65(Ts-CH₃), 29.66(CH), 52.09(C-6), 61.45(CH₂CH₃), 72.89(C-4), 78.63(C-5), 82.66(C-3), 127.79(C, Ts), 128.36(C, C, Ts), 130.48(C, C, Ts), 133.65(C, Ts), 137.37(C-1), 145.67(C-2), 166.02(COCH₂CH₃), 170.74(COCH₃) ppm.

ESI+490.26(M+Na).

### EXAMPLE 12

### Ethyl (3R,4R,5S)-4-acetamido-5-azido-3-(3-pentyloxy)cyclohex-1-ene-carboxylate 17

Sodium azide (0.015 g, 0.16 mmol) was added to a solution of 16 (0.015 g, 0.033 mmol) in dimethylformamide (1 mL) and heated at 75° C. for 6 h. The reaction mixture was concentrated in vacuo to dryness and dichloromethane (2 mL) and water (0.5 mL) were added, the phases were separated, and the organic phase was concentrated to dryness and the residue purified by chromatography to obtain 17 as crystalline solid (0.008 g, 74%).

¹H NMR (300 MHz-CDCl₃), δ 0.92(m, 6H, CHCH₂CH₃), 1.31(t, 3H, CH₂CH₃), 1.47-1.55 (m, 4H, CHCH₂CH₃), 2.05(s, 3H, COCH₃), 2.18-2.32(m, 1H, ring CH₂), 2.79-2.94(dd, 1H, ring CH₂), 3.22-3.39(m, 2H, H-4, H-5), 4.21(q, 2H, CH₂CH₃), 4.32(m, 1H, CH₂CHCH₂), 4.56-4.62(m, 1H, H-3), 4.69-4.81(bd, 1H, NH), 6.79-6.82(s, 1H, H-2) ppm.

¹³C NMR (300 MHz-CDCl₃) δ 9.26(CH₃), 9.54(CH₃), 14.15(CH₃), 23.51 (CH₂), 25.58(CH₂), 26.24(COCH₃), 30.49(CH), 57.29(C-6), 57.83(C-4), 61.03(CH₂CH₃), 73.52(C-5), 82.03(C-3), 128.09(C-1), 137.98(C-2), 165.79(COCH₂CH₃), 171.13(COCH₃) ppm.

### EXAMPLE 13

### Ethyl (3R,4R,5S)-4-acetamido-5-amino-3-(3-pentyloxy)cyclohex-1-ene-carboxylate phosphate salt 18

The title compound was prepared according to the procedure given in Journal of Organic Chemistry (1998) 63, 4545-4550 (compound 2) and the ¹H NMR spectrum is given below.

To a solution of 17 (20 mg, 0.06 mmol) in EtOH (2 mL), was added Lindlar's catalyst (5 mg) and the mixture was stirred under a hydrogen atmosphere for 19 h. After completion of the reaction, the solid was removed by filtration and washed with EtOH (2 mL). The filtrate was concentrated under vacuum and the crude product was purified by column chromatography and concentrated to dryness. The product was taken in EtOH (2 mL) and H₃PO₄ (5 mg) in EtOH (1 mL) was added and crystallized to obtain 18 as white solid (16 mg, 66%).

¹H NMR (300 MHz-CDCl₃), δ 0.79-0.92(m, 6H, CHCH₂CH₃), 1.28-1.34(t, 3H, CH₂CH₃), 1.43-1.65(m, 4H, CHCH₂CH₃), 2.09(s, 3H, COCH₃), 2.46-2.59(m, 1H, ring CH₂), 2.79-3.01(dd, 1H, ring CH₂), 3.51-3.62(m, 2H, H-4, H-5), 4.07(m, 1H, CH₂CHCH₂), 4.28(q, 2H, CH₂CH₃), 4.34(d, 1H, H-3), 6.87(s, 1H, H-2) ppm.

### EXAMPLE 14

### Ethyl (3S,4R,5R)-4-azido-5-(tert.butyldiphenyl)silyloxy-3-tosyloxycyclohex-1-ene-carboxylate 19

Tert-butyldiphenylsilyl chloride (TBDPS—Cl) (0.5 mL, 1.95 mmol) was added for 3 min to a solution of 11a (0.57 g, 1.5 mmol), triethylamine (0.52 mL, 3.74 mmol), and 4-dimethylaminopyridine (5 mg) in dichloromethane (10 mL) at room temperature and the mixture was stirred a further 24 h. 5% Aq. sodium bicarbonate solution (3 mL) was added and the organic layer was separated, concentrated to dryness and the residue purified by chromatography to obtain 19 as crystalline solid (0.8 g, 98%).

¹H NMR (300 MHz-CDCl₃), δ 1.08(s, 9H, t-Bu), 1.22(t, 3H, CH₂CH₃), 2.32-2.52(m, 5H, Ts-CH₃, ring CH₂), 3.78-3.89(m, 2H, H-4, H-5), 5.01(m, 1H, H-3), 6.37(s, 1H, H-2), 7.38-7.55(m, 8H, Ts, Ph), 7.67(m, 4H, Ph), 7.79(d, 2H, Ts) ppm.

### EXAMPLE 15

### Ethyl (3S,4R,5R)-4-amino-5-(tert.butyldiphenyl)silyloxy-3-tosyloxycyclohex-1-ene-carboxylate 20

1M Trimethylphosphine in toluene (1.2 mL, 1.21 mmol) was added for 3 min to a solution of 19 (0.68 g, 1.1 mmol) in anhydrous tetrahydrofuran (10 mL) at 0° C. then, after 10 min, the light yellow mixture was stirred at room temperature for 1 h. 1M Trimethylphosphine in toluene (0.22 mL, 0.22 mmol) was added and stirring continued for 30 min. where up water (0.2 mL) was added and heated at 45° C. for 45 min. The reaction mixture was concentrated to dryness and the residue purified by chromatography to obtain 20 as yellow oily compound (0.42 g, 65%).

¹H NMR (300 MHz-CDCl₃), δ 1.05(s, 9H, t-Bu), 1.19(t, 3H, CH₂CH₃), 2.28-2.38(dd, 1H, ring CH₂), 2.44-2.59(m, 4H, Ts-CH₃, ring CH₂), 3.34(bs, 1H, H-4), 3.83(m, 1H, H-5), 4.08-4.17(m, 2H, CH₂CH₃), 4.95(s, 1H, H-3), 6.42(s, 1H, H-2), 7.29-7.52(m, 8H, Ts, Ph), 7.62(m, 4H, Ph), 7.77(d, 2H, Ts) ppm.

### EXAMPLE 16

### Ethyl (3S,4R,5R)-3,4-imino-5-(tert.butyldiphenyl)silyloxycyclohex-1-ene-carboxylate 22

Lithium bromide (0.3 g, 3.54 mmol) was added to a solution of 20 in ethanol (5 mL) at 0° C., stirred for 16 h at room temperature and concentrated to dryness. Dichloromethane (10 mL) and water (2 mL) were added and the mixture vigorously stirred for 5 min. and the organic layer was separated and concentrated to dryness to afford 21.

¹H NMR (300 MHz-CDCl₃), δ 1.06(s, 9H, t-Bu), 1.19(t, 3H, CH₂CH₃), 2.48-(bs, 2H, ring CH₂), 3.14(dd, 1H, H-4), 4.06-4.19(m, 2H, CH₂CH₃), 4.29(m, 1H, H-5), 4.69(m, 1H, H-3), 6.97(bs, 1H, H-2), 7.28-7.81(m, 10H, Ph) ppm.

The crude 21 was dissolved in dichloromethane (2 mL) to which was added triethylamine (0.5 mL) and heated at 35° C. for 5 h. The reaction mixture was concentrated to dryness and the residue was purified by chromatography to yield 22 (0.04 g, 15%).

¹H NMR (300 MHz-CDCl₃), δ 1.07(s, 9H, t-Bu), 1.25(t, 3H, CH₂CH₃), 2.15-2.31(m, 1H, H-4), 2.39-2.52(m, 2H, ring CH₂), 3.79(dd, 1H, H-3), 4.09-4.25(m, 3H, CH₂CH₃, H-5), 7.02(t, 1H, H-2), 7.38-7.82(m, 10H, Ph) ppm.

¹³C NMR (300 MHz-CDCl₃) δ 14.20(CH₂CH₃), 19.21(C—(CH₃)₃), 26.92(C—(CH₃)₃), 28.87(C-6), 29.50(C-4), 38.68(C-3), 60.60(CH₂CH₃), 68.55(C-5), 127.65(Ph), 127.73(Ph), 129.72(H-1), 134.63(Ph), 136.76(C-2), 165.98(COCH₂CH₃) ppm.

ESI+444.32(M+Na).

### EXAMPLE 17

### Ethyl (3S,4R,5R)-3,4-acetylimino-5-(tert.butyldiphenyl)silyloxycyclohex-1-ene-carboxylate 23

Acetyl chloride (6.3 μL, 0.09 mmol) was added to a solution of 22 in dichloromethane (2 mL), triethylamine (20 μL, 0.14 mmol) at 0° C., and the mixture was stirred for 30 min., concentrated to dryness and the residue was purified by chromatography to obtain 23 (0.02 g, 52%).

¹H NMR (300 MHz-CDCl₃), δ 1.12(s, 9H, t-Bu), 1.24(t, 3H, CH₂CH₃), 2.19(s, 3H, COCH₃), 2.21-2.33(m, 1H, H-4), 2.65-2.79(m, 2H, ring CH₂), 3.01(t, 1H, H-3), 3.94-4.03(m, 1H, H-5), 4.04-4.22(m, 2H, CH₂CH₃), 6.95(t, 1H, H-2), 7.39-7.79(m, 10H, Ph) ppm.

¹³C NMR (300 MHz-CDCl₃) δ 14.14(CH₂CH₃), 19.17(C—(CH₃)₃), 23.38(COCH₃), 26.92(C—(CH₃)₃), 26.95(C—(CH₃)₃), 29.43(C-6), 34.75(C-4), 42.89(C-3), 60.78(CH₂CH₃), 67.45(C-5), 127.79(Ph), 129.99(Ph), 132.58(H-1), 133.59(H-2), 135.79(Ph) 165.61(COCH₂CH₃), 182.06(COCH₃) ppm.

ESI+464.31(M+H).

As many changes can be made to the examples which exemplify the invention without departing from the scope of the invention, it is intended that all matter contained herein be considered illustrative of the invention and not in a limiting sense.

The embodiments of the invention in which an exclusive property or privilege is claimed are as follows:

1. A process for the preparation of compound 6, 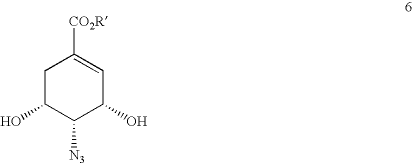
comprising the steps of:
i) converting a compound 5 to compound 6 by base treatment, 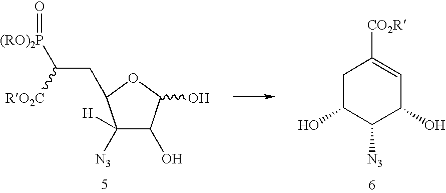
wherein R and R′ are selected from the group consisting of substituted or unsubstituted linear or branched C1 to C6 alkyl, C6 to C9 aryl, and C7 to C10 aralkyl.

2. A process according to claim 1, wherein compound 5 is prepared by hydrolyzing a compound 4, 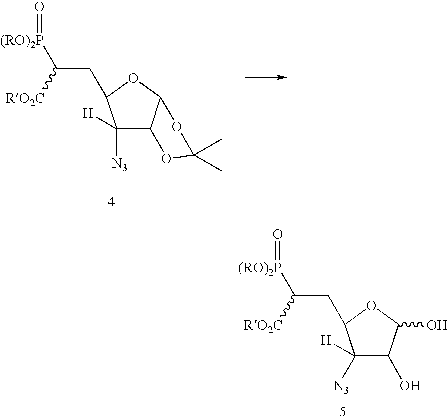
wherein R and R are selected from the group consisting of substituted or unsubstituted linear or branched C1 to C6 alkyl, C6 to C9 aryl, and C7 to C10 aralkyl.

3. A process according to claim 2, wherein compound 4 is prepared by removing a leaving group on a compound 2 by using an anion of a trialkylphosphonacetate,

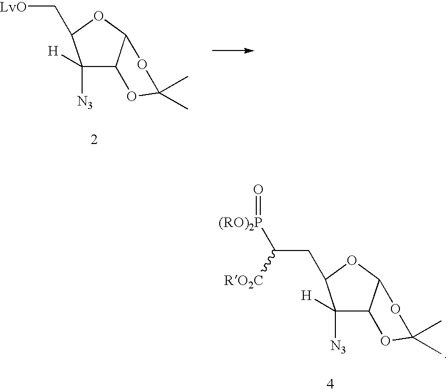

4. A process according to claim 3, wherein compound 2 is prepared by converting a hydroxyl group on a compound 1 to a leaving group,

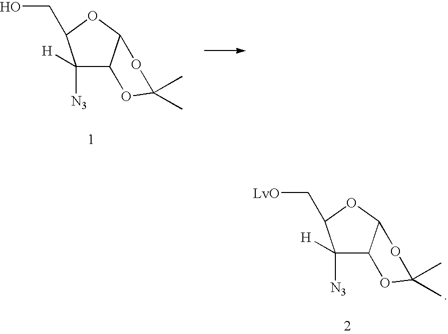

5. A process according to claim 4, wherein compound 1 is prepared by either: (a) converting D-glucose to compound 1 by acetonide formation, triflation, azide displacement, acetonide hydrolysis, periodate cleavage, and reduction;
(b) or converting D-xylose to compound 1 by diacetonide formation, selective acetonide hydrolysis, protection of the primary hydroxyl group, triflation, azide displacement, and removal of the primary hydroxyl protecting group,

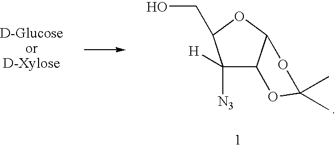

6. A process for the preparation of compound 10, 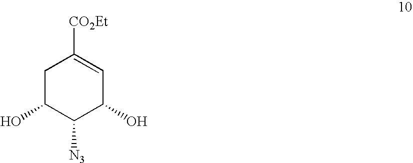
comprising the step of treating a compound 9 with a base to yield compound 10,

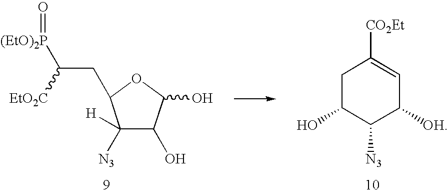

7. A process according to claim 6, wherein compound 9 is prepared by hydrolyzing a compound 8 using an acid,

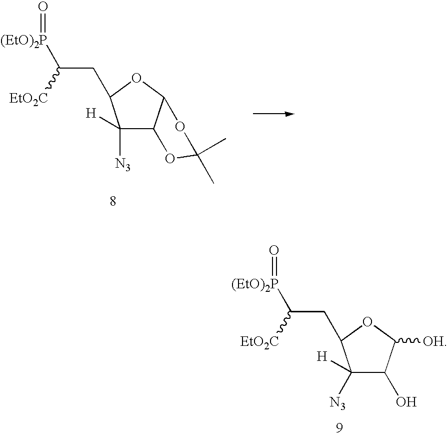

8. A process according to claim 7, wherein compound 8 is prepared by displacing a triflate group on a compound 7 using an anion of diethylphosphonoacetate,

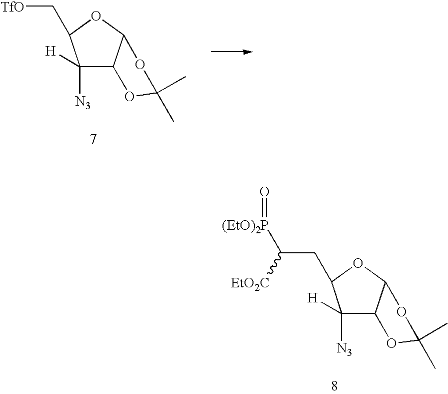

9. A process according to claim 8, wherein compound 7 is prepared by triflation of a primary hydroxyl on a compound 1,

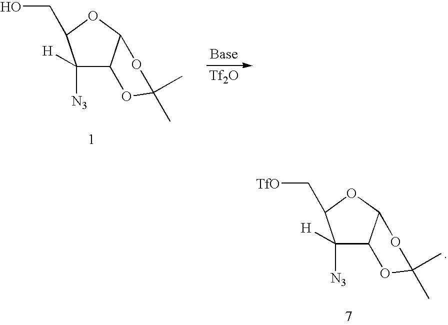

10. A process according to claim 9, wherein compound 1 is prepared by either converting:
(a) D-glucose to compound 1 by acetonide formation, triflation, azide displacement, acetonide hydrolysis, periodate cleavage, and reduction;
(b) or converting D-xylose to compound 1 by diacetonide formation, selective acetonide hydrolysis, protection of the primary hydroxyl group, triflation, azide displacement, and removal of the primary hydroxyl protecting group,

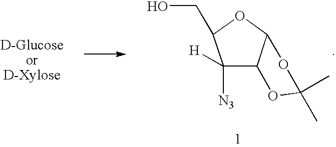

11. A process for the preparation of Oseltamivir Phosphate 18 comprising the steps of:
i) converting compound 17 to Oseltamivir phosphate 18 by azide reduction and salt formation; 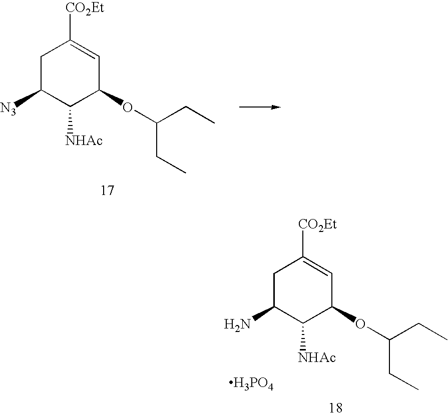
ii) converting compound 16 to compound 17 by azide displacement; 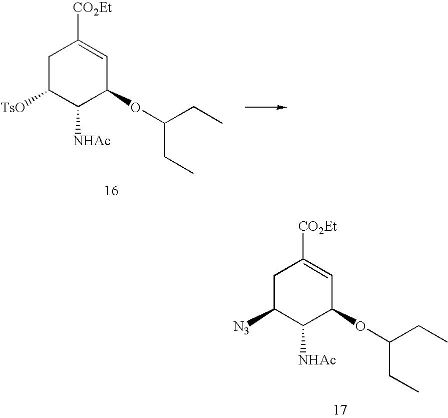
iii) converting compound 15 to compound 16 by Lewis-acid mediated aziridine ring opening in the presence of 3-pentanol; 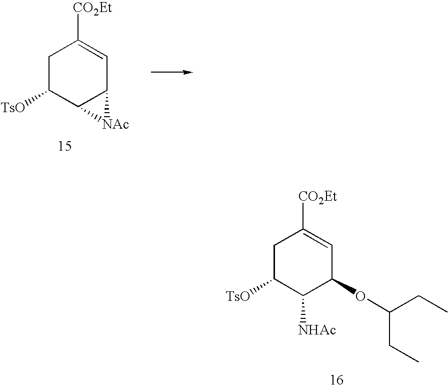
iv) converting compound 14 to compound 15 by acetylation; 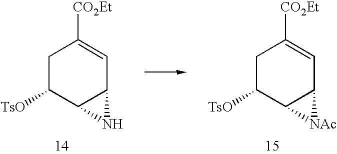
v) converting compound 13 to compound 14 by base-mediated aziridine ring formation; 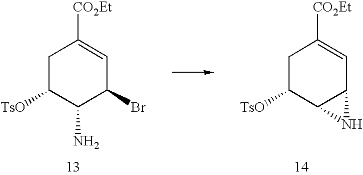
vi) converting compound 12 to compound 13 by bromide displacement; 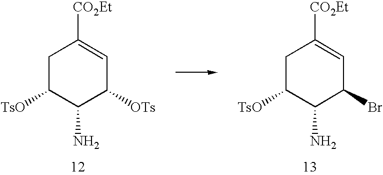
vii) converting compound 1b to compound 12 by trialkylphosphine mediated azide reduction; 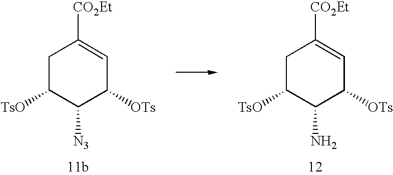
viii) converting compound 10 to compound 11b by ditosylation; 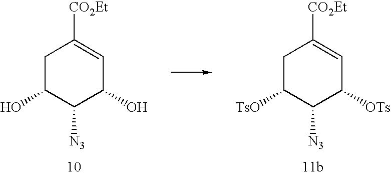
ix) converting compound 9 to compound 10 by base treatment to achieve intramolecular cyclization; 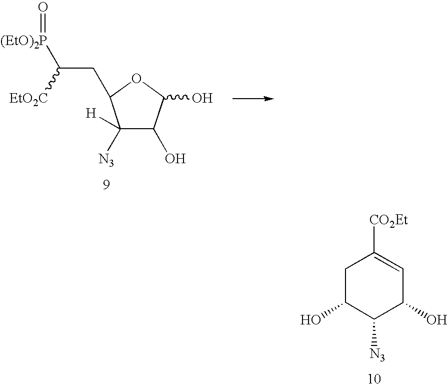
x) converting compound 8 to compound 9 by acid hydrolysis; 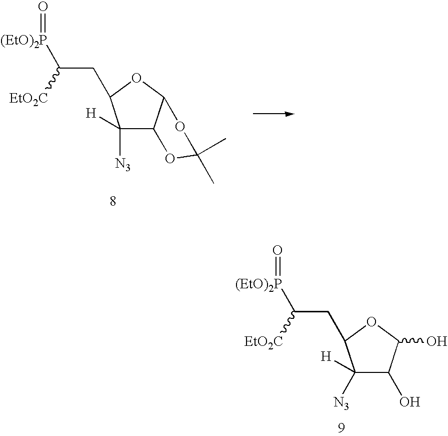
xi) converting compound 7 to compound 8 by displacement of the triflate group using the anion of dialkylphosphonoacetate; 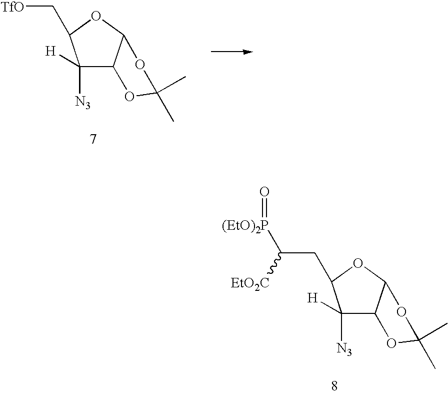
xii) converting compound 1 to compound 7 by triflation of the primary hydroxyl; 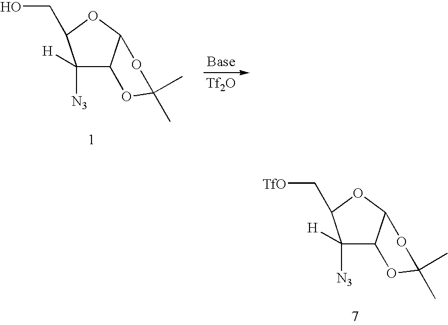
and either
xiii) (a) converting D-glucose to compound 1 by acetonide formation, triflation, azide displacement, acetonide hydrolysis, periodate cleavage, and reduction,
(b) or converting D-xylose to compound 1 by diacetonide formation, selective acetonide hydrolysis, protection of the primary hydroxyl group, triflation, azide displacement, and removal of the primary hydroxyl protecting group.

12. A process for the preparation of Oseltamivir Phosphate 18 comprising the steps of:
i) converting compound 17 to Oseltamivir phosphate 18 by azide reduction and salt formation; 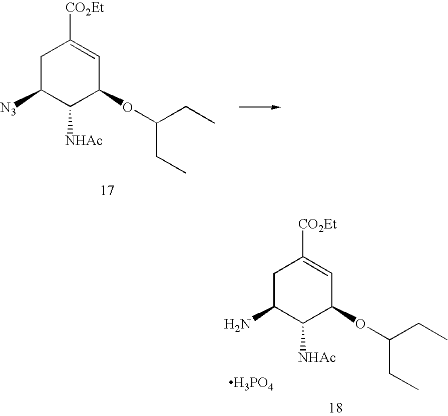
ii) converting compound 16 to compound 17 by azide displacement; 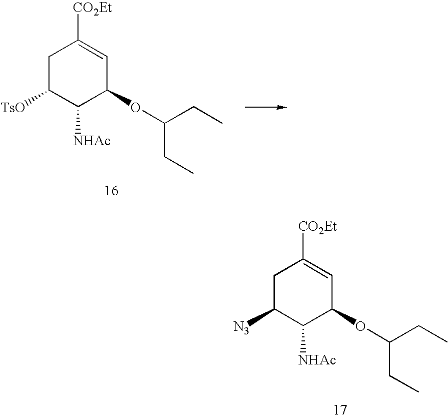
iii) converting compound 23 to compound 16 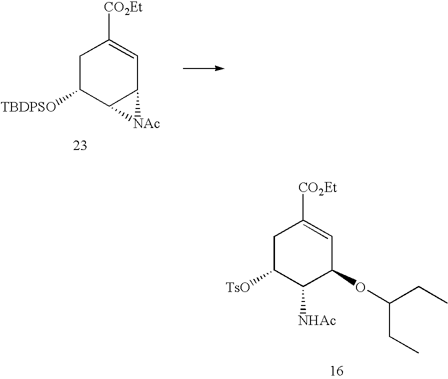
by:
1) a Lewis acid-mediated ring-opening of the acetylaziridine ring of compound 23 in the presence of 3-pentanol;
2) followed by a deprotecting step using a deprotecting agent; and
3) followed by a tosylation step;
iv) compound 23 is prepared by acetylating a compound 22 using an acetylating agent in the presence of a base; 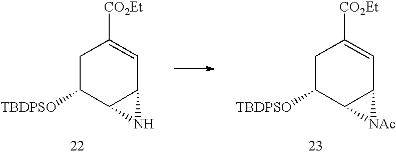
v) compound 22 is prepared by treating a compound 21 with a base in a suitable solvent; 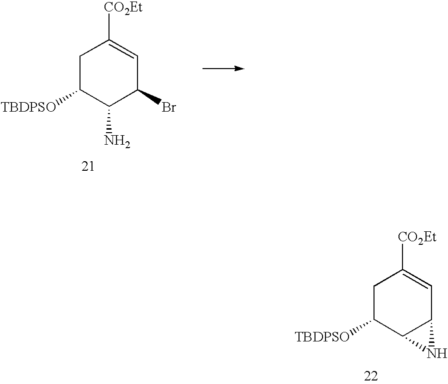
vi) compound 21 is prepared by treating a compound 20 with lithium bromide in alcohol; 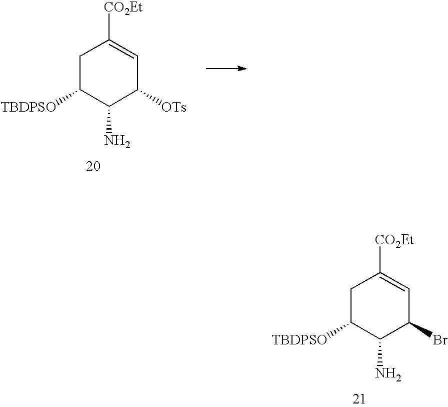
vii) compound 20 is prepared by reducing a compound 19 using a trialkylphosphine in tetrahydrofuran containing water; 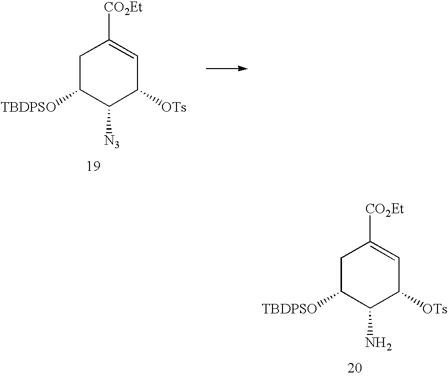
viii) compound 19 is prepared by treating a compound 11a with a silylating reagent; 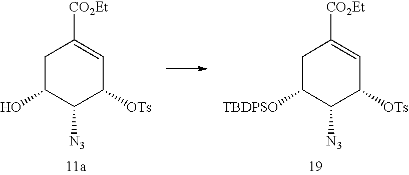
ix) compound 10 is converted to compound 11a by tosylation; 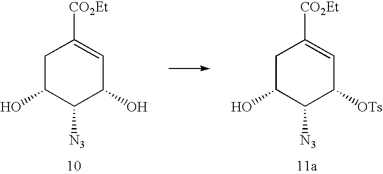
x) converting compound 9 to compound 10 by base treatment to achieve intramolecular cyclization; 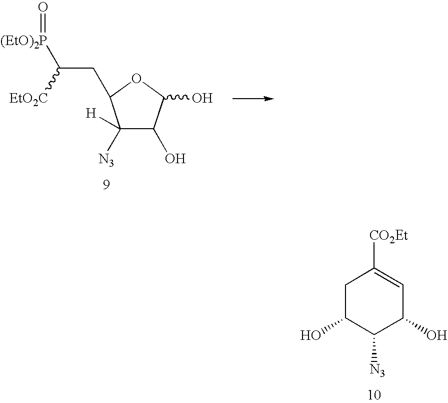
xi) converting compound 8 to compound 9 by acid hydrolysis; 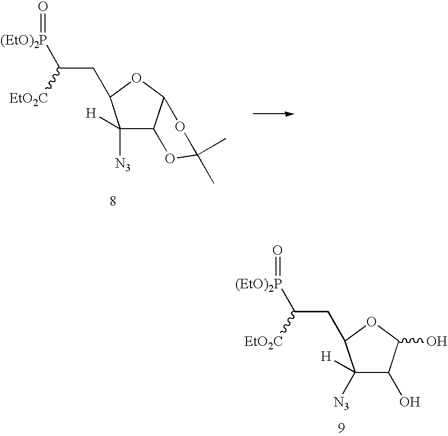
xii) converting compound 7 to compound 8 by displacement of the triflate group using the anion of dialkylphosphonoacetate; 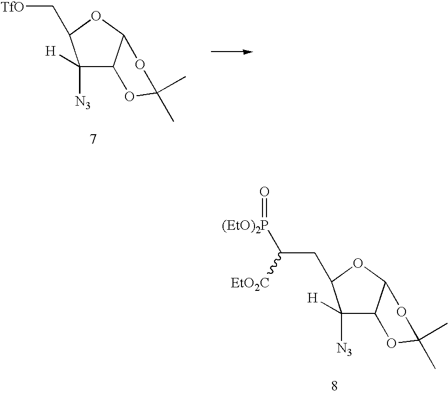
xiii) converting compound 1 to compound 7 by triflation of the primary hydroxyl; 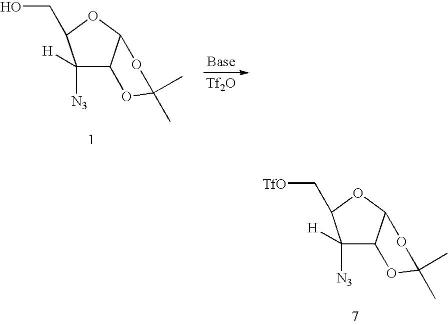
and either
xiv) (a) converting D-glucose to compound 1 by acetonide formation, triflation, azide displacement, acetonide hydrolysis, periodate cleavage, and reduction;
(b) or converting D-xylose to compound 1 by diacetonide formation, selective acetonide hydrolysis, protection of the primary hydroxyl group, triflation, azide displacement, and removal of the primary hydroxyl protecting group.

13. A process for the preparation of Oseltamivir Phosphate 18 comprising the steps of
i) converting compound 27 to compound 28 to compound 18 by: 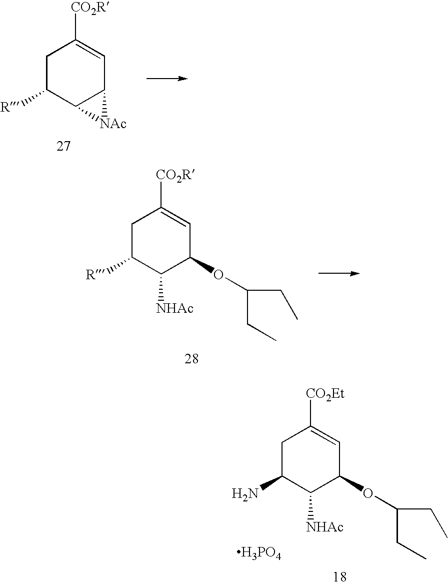
1) Lewis-acid mediated ring opening in the presence of 3-pentanol;
2) azide displacement;
3) azide reduction; and
4) salt formation;
ii) converting a compound 26 to a compound 27 by:
1) base mediated aziridine ring formation, and
2) acetylation using an acetylating agent, 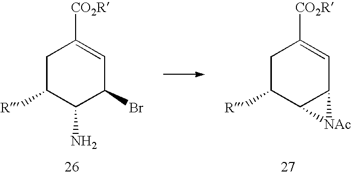
iii) treating a compound 25 to a bromide displacement step to yield compound 26; 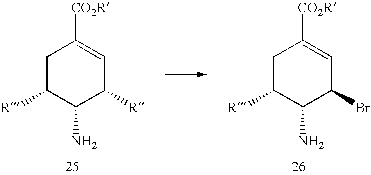
iv) treating a compound 24 to an azide reduction step to yield compound 25; 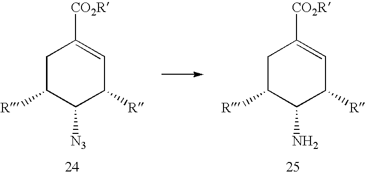
v) converting a compound 10 to a compound 24 by protection of the alcoholic groups; 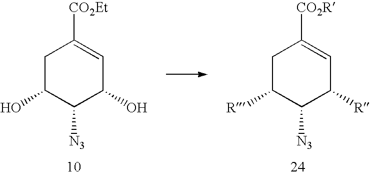
vi) converting compound 9 to compound 10 by base treatment to achieve intramolecular cyclization; 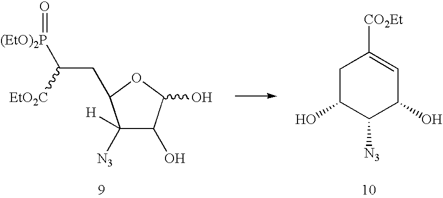
vii) converting compound 8 to compound 9 by acid hydrolysis; 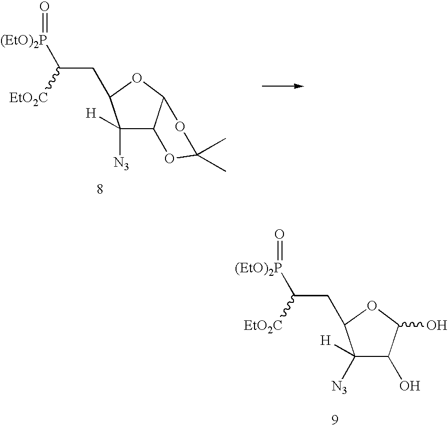
viii) converting compound 7 to compound 8 by displacement of the triflate group using the anion of dialkylphosphonoacetate; 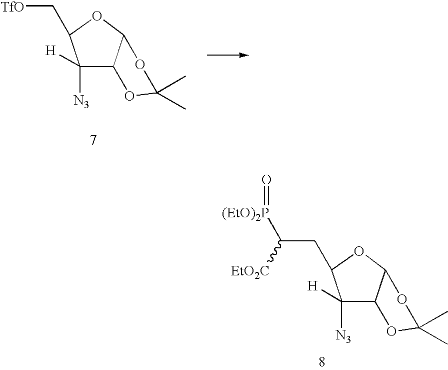
ix) converting compound 1 to compound 7 by triflation of the primary hydroxyl; 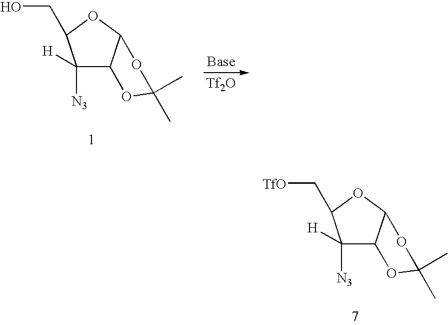
and either
x) (a) converting D-glucose to compound 1 by acetonide formation, triflation, azide displacement, acetonide hydrolysis, periodate cleavage, and reduction;
(b) or converting D-xylose to compound 1 by diacetonide formation, selective acetonide hydrolysis, protection of the primary hydroxyl group, triflation, azide displacement, and removal of the primary hydroxyl protecting group.

14. A process for the preparation of compound 18, 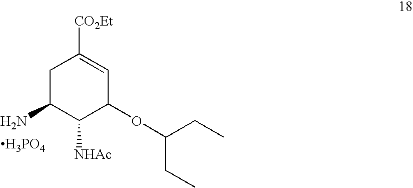
comprising the steps of:
i. converting a compound 16 to a compound 17 by azide displacement; and
ii. converting compound 17 to compound 18 by azide reduction and salt formation,

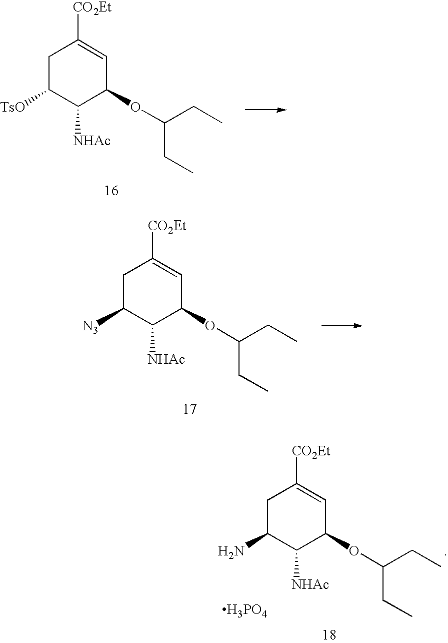

15. A process according to claim 14, wherein compound 16 is prepared by converting a compound 15 by a Lewis-acid mediated aziridine ring opening reaction in the presence of 3-pentanol,

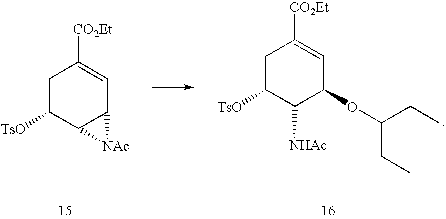

16. A process according to claim 15, wherein compound 15 is prepared by acetylating a compound 14 by using an acetylating agent,

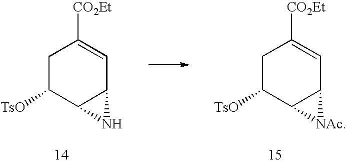

17. A process according to claim 16, wherein compound 14 is prepared by treating a compound 13 to a base-mediated aziridine ring formation,

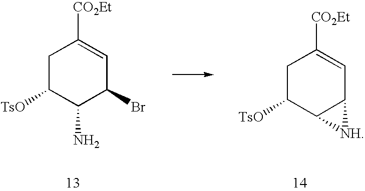

18. A process according to claim 17, wherein compound 13 is prepared by treating a compound 12 to a bromide displacement step,

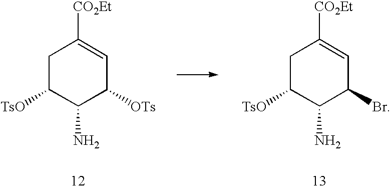

19. A process according to claim 18, wherein compound 12 is prepared by treating a compound 11b to a trialkylphosphine mediated azide reduction step,

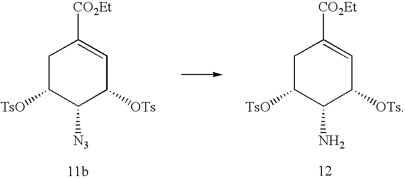

20. A process according to claim 19, wherein compound 11b is prepared by ditosylating a compound 10,

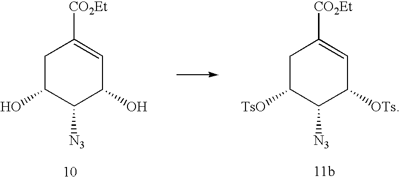

21. A process for the preparation of compound 16, 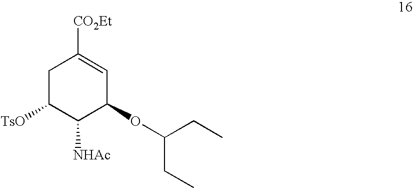
comprising the steps of converting a compound 23 to compound 16,
i. by a Lewis acid-mediated ring-opening of the acetylaziridine ring of compound 23 in the presence of 3-pentanol;
ii. followed by a deprotecting step using a deprotecting agent, and
iii. followed by a tosylation step;

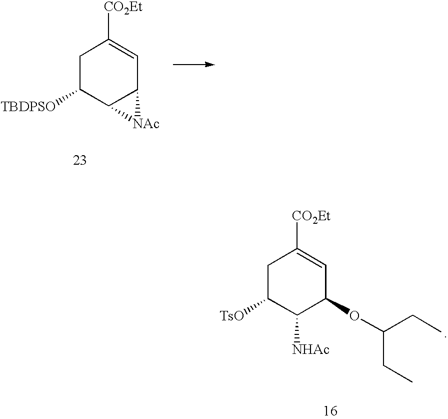

22. A process according to claim 21, wherein compound 23 is prepared by acetylating a compound 22 using an acetylating agent in the presence of a base,

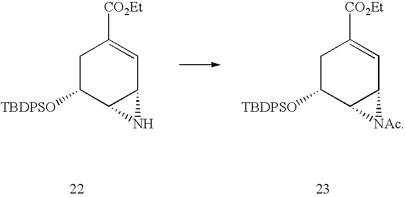

23. A process according to claim 22, wherein compound 22 is prepared by treating a compound 21 with a base in a suitable solvent,

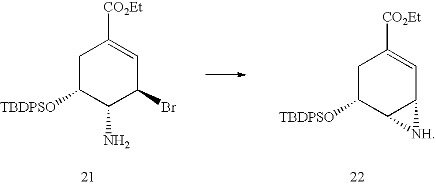

24. A process according to claim 23, wherein compound 21 is prepared by treating a compound 20 with lithium bromide in alcohol,

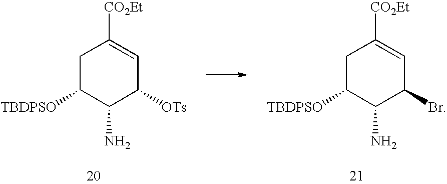

25. A process according to claim 24, wherein compound 20 is prepared by reducing a compound 19 using a trialkylphosphine in tetrahydrofuran containing water,

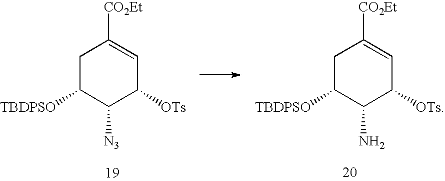

26. A process according to claim 25, wherein compound 19 is prepared by treating a compound 11a with a silylating reagent,

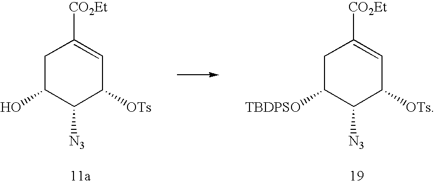

27. A process for the preparation of compound Oseltamivir 18, 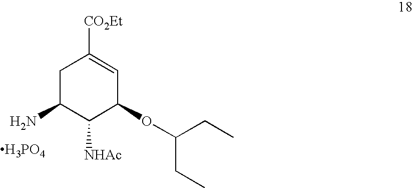
comprising the steps of converting compound 27 to compound 28 to compound 18 by:
i. Lewis-acid mediated ring opening in the presence of 3-pentanol;
ii. azide displacement;
iii. azide reduction; and
iv. salt formation; 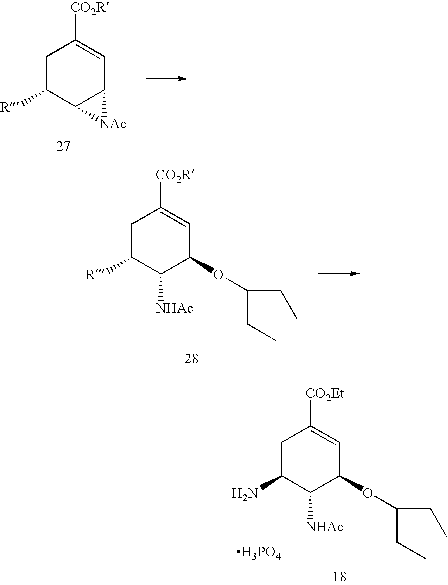
wherein R′ is selected from substituted or unsubstituted, linear or branched C1 to C6 alkyl, C6 to C9 aryl, or C7 to C10 aralkyl group R′″ is a leaving group selected from mesyloxy, trifloxy or tosyloxy or a protecting group selected from silyloxy groups.

28. A process according to claim 27, wherein compound 27 is prepared by treating a compound 26 to a
i. base mediated aziridine ring formation; and
ii. acetylation using an acetylating agent; 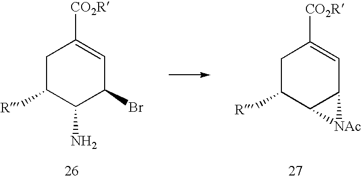
wherein R′ is selected from substituted or unsubstituted, linear or branched C1 to C6 alkyl, C6 to C9 aryl, or C7 to C10 aralkyl group and R′″ is a leaving group selected from mesyloxy, trifloxy or tosyloxy or a protecting group selected from silyloxy groups.

29. A process according to claim 28, wherein compound 26 is prepared by treating a compound 25 to a bromide displacement step, 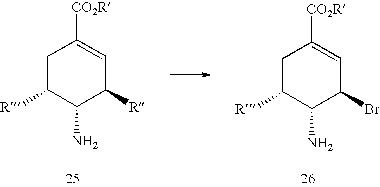
wherein R′ is selected from substituted or unsubstituted, linear or branched C1 to C6 alkyl, C6 to C9 aryl, or C7 to C10 aralkyl group, R″ is a leaving group selected from mesyloxy, trifloxy or tosyloxy, and R′″ is a leaving group the same as defined above for R″, or a protecting group selected from silyloxy groups.

30. The process according to claim 29, wherein R′ is selected from a substituted or unsubstituted C1 to C6 alkyl group, a substituted or unsubstituted C6 to C9 aryl group, or a substituted or unsubstituted C7 to C10 aralkyl group and R″ and R′″ are tosyloxy groups.

31. A process according to claim 30, wherein compound 25 is prepared by treating a compound 24 by trialkylphosphine mediated azide reduction step, 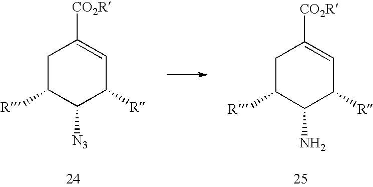
wherein R′ is selected from substituted or unsubstituted, linear or branched C1 to C6 alkyl, C6 to C9 aryl, or C7 to C10 aralkyl group, R″ is a leaving group selected from mesyloxy, trifloxy or tosyloxy, and R′″ is a leaving group the same as defined above for R″, or a protecting group selected from silyloxy groups.

32. The process according to claim 31, wherein R′ is selected from a substituted or unsubstituted C1 to C6 alkyl group, a substituted or unsubstituted C6 to C9 aryl group, or a substituted or unsubstituted C7 to C10 aralkyl group and R″ and R′″ are tosyloxy.

33. A process according claim 31, wherein compound 24 is prepared by treating a compound 6 by protection of the alcoholic groups, 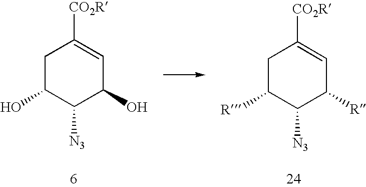
wherein R′ is selected from substituted or unsubstituted, linear or branched C1 to C6 alkyl, C6 to C9 aryl, or C7 to C10 aralkyl group, R″ is a leaving group selected from mesyloxy, trifloxy or tosyloxy, and R′″ is a leaving group the same as defined above for R″, or a protecting group selected from silyloxy groups.

34. The process according to claim 33, wherein R′ is selected from a substituted or unsubstituted C1 to C6 alkyl group, a substituted or unsubstituted C6 to C9 aryl group, or a substituted or unsubstituted C7 to C10 aralkyl group and R″ and R′″ are tosyloxy.

35. Compounds of formula 4: 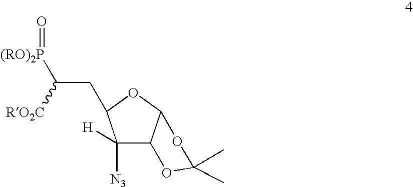
wherein R and R′ are independently selected from group consisting of substituted or unsubstituted linear or branched C1 to C6 alkyl, C6 to C9 aryl, and C7 to C10 aralkyl.

36. Compound according to claim 35, wherein R and R′ are both ethyl.

37. Compounds of formula 5: 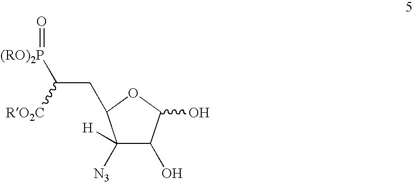
wherein R and R′ are independently selected from group consisting of substituted or unsubstituted linear or branched C1 to C6 alkyl, C6 to C9 aryl, and C7 to C10 aralkyl.

38. Compound according to claim 37, wherein R and R′ are both ethyl.

39. Compounds of formula 6: 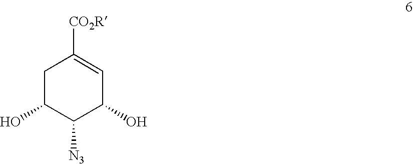
wherein R′ is selected from substituted or unsubstituted, linear or branched C1 to C6 alkyl, C6 to C9 aryl, and C7 to C10 aralkyl.

40. Compound according to claim 39, wherein R′ is ethyl.

41. Compounds of formula 24, 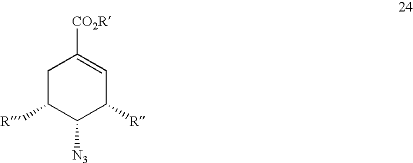
wherein R′ is selected from substituted or unsubstituted, linear or branched C1 to C6 alkyl, C6 to C9 aryl, or C7 to C10 aralkyl group, R″ is a leaving group selected from mesyloxy, trifloxy or tosyloxy, and R′″ is a leaving group as defined above for R″, or a protecting group selected from silyloxy groups.

42. Compound according to claim 41, wherein R′ is ethyl and R″ and R′″ are tosyloxy groups.

43. Compounds of formula 25, 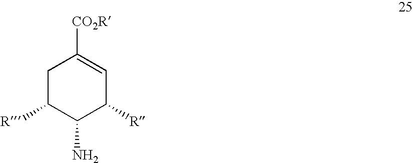
wherein R′ is selected from substituted or unsubstituted, linear or branched C1 to C6 alkyl, C6 to C9 aryl, or C7 to C10 aralkyl group, R″ is a leaving group selected from mesyloxy, trifloxy or tosyloxy, and R′″ is a leaving group the same as defined above for R″, or a protecting group selected from silyloxy groups.

44. Compound according to claim 43, wherein R′ is ethyl and R″ and R′″ are tosyloxy groups.

45. Compounds of formula 26, 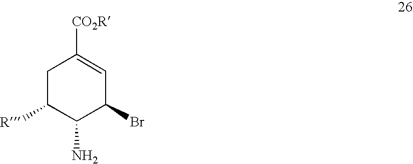
wherein R′ is selected from substituted or unsubstituted, linear or branched C1 to C6 alkyl, C6 to C9 aryl, or C7 to C10 aralkyl group and R′″ is a leaving group selected from mesyloxy, trifloxy or tosyloxy, or a protecting group selected from silyloxy groups.

46. Compound according to claim 45, wherein R′ is ethyl and R′″ is a tosyloxy group.

47. Compound according to claim 46, wherein R′ is ethyl and R′″ is a silyloxy protecting group.

48. Compounds of formula 27, 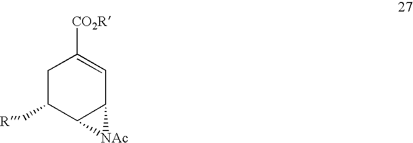
wherein R′ is selected from substituted or unsubstituted, linear or branched C1 to C6 alkyl, C6 to C9 aryl, or C7 to C10 aralkyl group and R′″ is a leaving group selected from mesyloxy, trifloxy or tosyloxy, or a protecting group selected from silyloxy groups.

49. Compound according to claim 48, wherein R′ is ethyl and R′″ is a tosyloxy group.

50. Compound according to claim 49, wherein R′ is ethyl and R′″ is a silyloxy protecting group.

51. Compounds of formula 28, 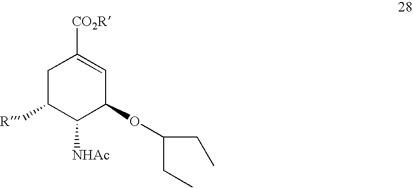
wherein R′ is selected from substituted or unsubstituted, linear or branched C1 to C6 alkyl, C6 to C9 aryl, or C7 to C10 aralkyl group and R′″ is a leaving group selected from mesyloxy, trifloxy or tosyloxy, or a protecting group selected from silyloxy groups.

52. Compound according to claim 51, wherein R′ is ethyl and R′″ is a tosyloxy group.

53. Compound according to claim 51, wherein R′ is ethyl and R′″ is a silyloxy protecting group.

54. Compound selected from the group consisting of:
Ethyl (3S,4R,5R)-4-azido-5-(tert.butyldiphenyl)silyloxy-3-tosyloxycyclohex-1-ene-carboxylate 19;
Ethyl (3S,4R,5R)-4-amino-5-(tert.butyldiphenyl)silyloxy-3-tosyloxycyclohex-1-ene-carboxylate 20;
Ethyl (3S,4R,5R)-3,4-imino-5-(tert.butyldiphenyl)silyloxycyclohex-1-ene-carboxylate 22;
Ethyl (3S,4R,5R)-3,4-acetylimino-5-(tert.butyldiphenyl)silyloxycyclohex-1-ene-carboxylate 23;
Ethyl (3S,4R,5R)-4-azido-5-hydroxy-3-tosyloxycyclohex-1-ene-carboxylate 11a;
Ethyl (3S,4R,5R)-4-azido-3,5-ditosyloxycyclohex-1-ene-carboxylate 11b;
Ethyl (3S,4R,5R)-4-amino-3,5-ditosyloxycyclohex-1-ene-carboxylate 12;
Ethyl (3R,4R,5R)-4-amino-3-bromo-5-tosyloxycyclohex-1-ene-carboxylate 13;
Ethyl (3S,4R,5R)-3,4-imino-5-tosyloxycyclohex-1-ene-carboxylate 14;
Ethyl (3S,4R,5R)-3,4-acetylimino-5-tosyloxycyclohex-1-ene-carboxylate 15;
Ethyl (3R,4R,5R)-4-acetamido-3-(3-pentyloxy)-5-tosyloxycyclohex-1-ene-carboxylate 16;
3-Azido-3-deoxy-1,2-O-isopropylidene-5-O-trifluoromethanesulfonyl-α-D-ribofuranose 7;
Ethyl (3-azido-3-deoxy-5,6-dideoxy-6R/S-diethoxyphosphoryl-1,2-O-isopropylidene-α-D-ribo-heptofuranose)uronate 8;
Ethyl (3-azido-3-deoxy-5,6-dideoxy-6R/S-diethoxyphosphoryl-α/β-D-ribo-heptofuranose)uronate 9; and
Ethyl (3S,4R,5R)-4-azido-3,5-dihydroxycyclohex-1-ene-carboxylate 10.
